(19) United States
(12) Patent Application Publication
(10) Pub. No.: US 2003/0129171 A1
(43) Pub. Date: Jul. 10, 2003

(54) Double-muscling in mammals

(76) Inventors: Luc Grobet, Esneux (BE); Michel Georges, Villers-aux-Tours (BE); Dominique Poncelet, Tongeren (BE)

Correspondence Address: Michael A. Slavin, Esq., McHale & Slavin, P.A., Suite 402, 4440 PGA Boulevard, Palm Beach Gardens, FL, 33410, US

(21) Appl. No.: 10/251,115

(22) Filed: Sep. 20, 2002

Related U.S. Application Data
(63) Continuation of application No. 09/007,761, filed on Jan. 15, 1998, now abandoned.
Application No. 09/007,761 is a continuation-in-part of application No. 08/891,789, filed on Jul. 14, 1997, now Pat. No. 6,103,466.

(51) Int. Cl. C12Q 1/68; A01K 67/027; A61K 48/00

(52) U.S. Cl. 424/93.21; 435/6; 514/44; 800/15

(57) ABSTRACT

A gene (cDNA) encoding a bovine myostatin protein. The nucleic acid coding sequence is identified as SEQ ID NO:1 and the protein sequence is identified as SEQ ID NO:2. A mutant gene (SEQ ID NO:3) in which the coding sequence lacks an 11-base pair consecutive sequence (SEQ ID NO:11) of the sequence encoding bovine protein having myostatin activity has been sequenced. It has been shown that cattle of the Belgian Blue breed homozygous for the mutant gene lacking myostatin activity are double-muscled. A method for determining the presence of muscular hyperplasia in a mammal is described. The method includes obtaining a sample of material containing DNA from the mammal and ascertaining whether a sequence of the DNA encoding (a) a protein having biological activity of myostatin, is present, and whether a sequence of the DNA encoding (b) an allelic protein lacking the activity of (a), is present. The absence of (a) and the presence of (b) indicates the presence of muscular hyperplasia in the mammal.

### RELATED APPLICATION

This application is a Continuation-in-Part Application of prior U.S. patent application Ser, No. 08/891,789, filed Jul. 14, 1997, the specification of which is incorporated herein by reference.

### FIELD OF THE INVENTION

This invention relates to factors affecting muscle development in mammals, especially livestock. In particular, this invention relates to the cloning of the myostatin gene, a member of the TGF-β superfamily, its involvement in muscular hyperplasia in livestock, and a method for determining myostatin genotypes.

### DESCRIPTION OF RELATED ART

The TGF-β superfamily consists of a group of multifunctional polypeptides which control a wide range of differentiation processes in many mammalian cell types. GDF-8 is a member of the TGF-β superfamily. All members of this superfamily share a common structure including a short peptide signal for secretion and an N-terminal peptide fragment that is separated from the bioactive carboxy-terminal fragment by proteolytic cleavage at a highly conserved proteolytic cleavage site. The bioactive carboxy-terminal domain is characterized by cysteine residues at highly conserved positions which are involved in intra- and intermolecular disulfide bridges. The functional molecules are covalently linked (via a S—S bond) dimers of the carboxy-terminal domain (Masterson et al., 1996).

Recently, it was reported that mice deficient in the gene coding for GDF-8 were characterized by a generalized muscular hyperplasia (McPherron et al., 1997). The GDF-8 deficient mice were produced by gene targeting using homologous recombination in embryonic stem cells, a method referred to as “gene knock-out”. The murine generalized muscular hyperplasia appeared to be very similar in its expression to the muscular hyperplasia characterizing “double-muscled” cattle. This observation raised the intriguing possibility that the bovine gene coding for GDF-8 (i.e. the bovine evolutionary homologue of the mouse GDF-8 gene) is involved in the bovine double-muscling phenotype. It also raised the possibility that the human gene coding for GDF-8 (i.e. the human evolutionary homologue of the mouse GDF-8 gene) is involved in regulating muscular development in humans, specifically skeletal muscle genesis. Isolation of the human GDF-8 gene may have therapeutic uses/applications in the treatment of musculodegenerative diseases through upgrading or downgrading the expression of GDF-8.

The occurrence of animals characterized by a distinct generalized muscular hypertrophy, commonly known as “double-muscled” animals, has been reported in several cattle breeds around the world. The first documented description of double-muscled cattle dates back as early as 1807 (Culley, 1807). One of the breeds in which this characteristic has been most thoroughly analyzed is the Belgian Blue Cattle Breed (“Belgian Blue Breed”). This is one of the only breeds where the double-muscled trait has been systematically selected for, and where the double-muscled phenotype is virtually fixed. A comparison of double-muscled and conventional animals within the Belgian Blue Breed, showed an increase in muscle mass by 20% on average, while all other organs were reduced in size (Hanset, 1986 and 1991). The muscular hypertrophy was shown to be an histological hyperplasia affecting primarily superficial muscles, accompanied by a 50% reduction in total lipid content and a reduction in connective tissue fraction as measured by hydroxyproline content (Hanset et al., 1982). Double-muscled animals were shown to have a reduced feed intake with improved feed conversion ratio (Hanset et al., 1987). An important economic benefit of double-muscled animals, in contrast to conventional animals, is the substantial increase in selling price and net income for the farmer (Hanset et al., 1987).

One of the most thorough series of studies on double-muscling is that of Hanset and colleagues in the Belgian Blue Breed. Objective criteria of muscular development, such as dressing-out percentage, lean and fat percentage, plasma and red cell creatine and creatinine concentrations, were measured on nearly 150 randomly selected animals raised in standardized conditions. These studies clearly revealed abnormal, bimodal distributions of the double-muscled phenotype and objectively confirmed the visual classification traditionally performed by breeders on double-muscled and conventional animals. The phenotypic distribution was resolved using a maximum likelihood procedure into two component normal populations with a common variance which revealed mean differences of three to four standard deviations depending on the trait. This suggested the presence of an allele having a major effect on muscular development with a population frequency close to 50% (Hanset and Michaux, 1985b). The most convincing evidence in favour of such an allele, however, came from experimental crosses involving double-muscled Belgian Blue sires and Holstein Friesian dairy cows (the latter animals having very poor muscular development). While F1 offspring showed a phenotypic distribution very similar to their Holstein Friesian dams, backcrossing these F1's to double-muscled sires produced a bimodal BC generation, clearly pointing towards the Mendelian segregation of a recessive “mh” (muscular hypertrophy) allele (Hanset and Michaux., 1985a).

The same kind of experimental crosses were subsequently used to perform a whole genome scan using a microsatellite based marker map. To perform the linkage analysis, animals were classified as double-muscled or conventional. Very significant Logarithm of the Odds scores (lodscores) were obtained on chromosome 2 (>17), and multi point linkage analysis positioned the mh locus at the centromeric end of this chromosome, at [2]centimorgan from the nearest microsatellite marker: TGLA44. The corresponding chromosomal region accounted for all the variance of the trait assumed to be fully penetrant in this experiment (Charlier et al., 1995).

In humans, genes coding for some forms of muscular abnormalities have been isolated, e.g. muscular dystrophy. The present invention provides for the gene which regulates the development of skeletal muscle only, as opposed to other types of muscle, e.g. smooth or cardiac muscle. The present invention may provide an understanding of the role of the GDF-8 gene or its receptor in the regrowth of skeletal muscle in humans which only undergo a hyperplasic response.

### SUMMARY OF THE INVENTION

The present inventors have identified and sequenced a gene (cDNA and genomic) encoding a bovine myostatin protein. The nucleic acid coding sequence is identified as SEQ ID NO: 1 and the protein sequence is identified as SEQ ID NO:2. The genomic bovine sequence is identified as SEQ ID NO:54. A mutant gene (SEQ ID NO:3) in which the coding sequence lacks an 11-base pair consecutive sequence (SEQ ID NO: 11) of the sequence encoding bovine protein having myostatin activity has been sequenced. It has been shown that cattle of the Belgian Blue breed homozygous for the mutant gene lacking myostatin activity are double-muscled. Other bovine mutations which lead to double-muscling in have also been determined, being identified herein as nt419(del7-ins10), Q204X, E226X and C313Y, respectively.

In one aspect, the present invention thus provides a method for determining the presence of muscular hyperplasia in a mammal. The method includes obtaining a sample of material containing DNA from the mammal and ascertaining whether a sequence of the DNA encoding (a) a protein having biological activity of myostatin, is present, and whether a sequence of the DNA encoding (b) an allelic protein lacking the activity of (a), is present. The absence of (a) and the presence of (b) indicates the presence of muscular hyperplasia in the mammal.

Of course, the mutation responsible for the lack of activity can be a naturally occurring mutation, as is the case for the Belgian Blue, Asturiana, Parthenaise or Rubia Gallega breeds, shown here.

The mammal can be a human, bovine, etc.

There are several methods known for determining whether a particular nucleotide sequence is present in a sample. A common method is the polymerase chain reaction. A preferred aspect of the invention thus includes a step in which ascertaining whether a sequence of the DNA encoding (a) is present, and whether a sequence of the DNA encoding (b) is present includes amplifying the DNA in the presence of primers based on a nucleotide sequence encoding a protein having biological activity of myostatin.

A primer of the present invention, used in PCR for example, is a nucleic acid molecule sufficiently complementary to the sequence on which it is based and of sufficient length to selectively hybridize to the corresponding portion of a nucleic acid molecule intended to be amplified and to prime synthesis thereof under in vitro conditions commonly used in PCR. Likewise, a probe of the present invention, is a molecule, for example a nucleic acid molecule of sufficient length and sufficiently complementary to the nucleic acid molecule of interest, which selectively binds under high or low stringency conditions with the nucleic acid sequence of interest for detection thereof in the presence of nucleic acid molecules having differing sequences.

In preferred aspects, primers are based on the sequence identified as SEQ ID NO:7 or SEQ ID NO:54.

In another aspect, the invention is a method for determining the presence of muscular hyperplasia in a mammal which includes obtaining a sample of material containing mRNA from the mammal. Such method includes ascertaining whether a sequence of the mRNA encoding (A) a protein having biological activity of myostatin, is present, and whether a sequence of the mRNA encoding (B) a protein at least partially encoded by a truncated nucleotide sequence corresponding to substantially the sequence of the mRNA and lacking the activity of (A), is present. The absence of (A) and the presence of (B) indicates the presence of muscular hyperplasia in the mammal.

The mRNA encoding (A) and the truncated sequence can correspond to alleles of DNA of the mammal.

Again, if an amplification method such as PCR is used in ascertaining whether a sequence of the mRNA encoding (A) is present, and whether a sequence of the mRNA encoding (B) is present, the method includes amplifying the mRNA in the presence of a pair of primers complementary to a nucleotide sequence encoding a protein having biological activity of myostatin. Each such primer can contain a nucleotide sequence substantially complementary, for example, to the sequence identified as SEQ ID NO:7. The truncated sequence can contain at least 50 consecutive nucleotides substantially corresponding to 50 consecutive nucleotides of SEQ ID NO:7, for example.

In another aspect, the invention is a method for determining the presence of muscular hyperplasia in a mammal which includes obtaining a tissue sample of containing mRNA of the mammal and ascertaining whether an mRNA encoding a mutant type myostatin protein lacking biological activity of myostatin is present. The presence of such an mRNA encoding a mutant type myostatin protein indicates the presence of muscular hyperplasia in the mammal.

In another aspect, the invention thus provides a method for determining the presence of muscular hyperplasia in a bovine animal. The method includes obtaining a sample of material containing DNA from the animal and ascertaining whether DNA having a nucleotide sequence encoding a protein having biological activity of myostatin is present. The absence of DNA having such a nucleotide sequence indicates the presence of muscular hyperplasia in the animal. Ascertaining whether DNA having a nucleotide sequence encoding a protein having biological activity of myostatin can include amplifying the DNA in the presence of primers based on a nucleotide sequence encoding a protein having biological activity of myostatin.

In particular, the method can be carried out using a sample from an animal in which such a bovine animal not displaying muscular hyperplasia is known to have a nucleotide sequence which is capable of hybridizing with a nucleic acid molecule having the sequence identified as SEQ ID NO:1 under stringent hybridization conditions.

It is possible that ascertaining whether DNA having a nucleotide sequence encoding a protein having biological activity of myostatin is present includes amplifying the DNA in the presence of primers based on a nucleotide sequence encoding the N-terminal and the C-terminal, respectively, of the protein having biological activity of myostatin.

Primers, say first and second primers, can be based on first and second nucleotide sequences encoding spaced apart regions of the protein, wherein the regions flank a mutation known to naturally occur and which when present in both alleles of a such an animal results in muscular hyperplasia.

It can also be that DNA of such an animal not displaying muscular hyperplasia contains a nucleotide sequence which hybridizes under stringent conditions with a nucleotide sequence encoding a protein having a sequence identified as SEQ ID NO:2 and the coding sequence of DNA of a such an animal displaying muscular hyperplasia is known to contain an 11-base pair deletion beginning at base pair no. 821 of the coding sequence, and said first primer is selected to be upstream of the codon encoding glutamic acid no. 275 and the second primer is selected to be downstream of the codon encoding aspartic acid no. 274.

Also, a DNA of such an animal not displaying muscular hyperplasia might contain a nucleotide sequence which hybridizes under stringent conditions with a nucleotide sequence encoding a protein having a sequence identified as SEQ ID NO:2. The coding sequence of DNA of such an animal displaying muscular hyperplasia might be known to contain an 11-base pair deletion beginning at base pair no. 821. A primer can be selected to span the nucleotide sequence including base pair nos. 820 and 821 of the DNA sequence containing the deletion.

The animal can be of the Belgian Blue breed.

In a particular aspect, ascertaining whether DNA having a nucleotide sequence encoding a protein having biological activity of myostatin is present includes amplifying the DNA in the presence of a primer containing at least a portion of a mutation known to naturally occur and which when present in both alleles of a said animal results in muscular hyperplasia.

In another aspect, the invention is a method for determining the presence of muscular hyperplasia in a bovine animal which includes obtaining a sample of the animal containing mRNA and ascertaining whether an mRNA encoding a protein having biological activity of myostatin is present in the sample. The absence of said mRNA indicates the presence of muscular hyperplasia in the animal.

A sample containing mRNA can be muscle tissue, particularly, skeletal muscle tissue.

In a particular aspect, the invention is a method for determining the presence of double muscling in a bovine animal, involving obtaining a sample of material containing DNA from the animal and ascertaining whether the DNA contains the nucleotide sequence identified as SEQ ID NO:11 in which the absence of the sequence indicates double muscling in the animal.

In a particular aspect, the animal is of the Belgian Blue breed.

In another aspect, the invention is a method for determining the myostatin genotype of a mammal, as may be desirable to know for breeding purposes. The method includes obtaining a sample of material containing nucleic acid of the mammal, wherein the nucleic acid is uncontaminated by heterologous nucleic acid; ascertaining whether the sample contains a (i) nucleic acid molecule encoding a protein having biological activity of myostatin; and ascertaining whether the sample contains an (ii) allelic nucleic acid molecule encoding a protein lacking biological activity of myostatin. The mammal can be bovine.

In another aspect, the subject is human and (i) includes a nucleic acid sequence substantially homologous (in the sense of identity) with the sequence identified as SEQ ID NO:7.

The invention includes a method of increasing muscle mass of a mammal having muscle cells in which myostatin is expressed, the method comprising administering to the mammal an effective amount of a nucleic acid molecule substantially complementary to at least a portion of mRNA encoding the myostatin and being of sufficient length to sufficiently reduce expression of the myostatin to increase the muscle mass. In a particular aspect, the mammal is bovine.

In another embodiment, the invention is a method of increasing muscle mass of a mammal, including administering to the mammal an effective amount of a nucleic acid molecule having ribozyme activity and a nucleotide sequence substantially complementary to at least a portion of mRNA encoding myostatin and being of sufficient length to bind selectively thereto to sufficiently reduce expression of the myostatin so as to increase the muscle mass.

The invention includes a diagnostic kit, for determining the presence of muscular hyperplasia in a mammal from which a sample containing DNA of the mammal has been obtained. The kit includes first and second primers for amplifying the DNA, the primers being complementary to nucleotide sequences of the DNA upstream and down stream, respectively, of a mutation in the portion of the DNA encoding myostatin which results in muscular hyperplasia of the mammal, wherein at least one of the nucleotide sequences is selected to be from a non-coding region of the myostatin gene. The kit can also includes a third primer complementary to a naturally occurring mutation of a coding portion of the myostatin gene.

A particular diagnostic kit, for determining the genotype of a sample of mammalian genetic material, particularly bovine material includes a pair of primers for amplifying a portion of the genetic material corresponding to a nucleotide sequence which encodes at least a portion of a myostatin protein, wherein a first of the primers includes a nucleotide sequence sufficiently complementary to a mutation of SEQ ID NO:1 to prime amplification of a nucleic acid molecule containing the mutation, the mutation being selected from the group of mutations resulting from: (a) deletion of 11 nucleotides beginning at nucleotide 821 of the coding portion of SEQ ID NO:1; (b) deletion of 7 nucleotides beginning at nucleotide 419 of the coding sequence and insertion of the sequence AAGCATACAA in place thereof; (c) deletion of nucleotide 204 of the coding sequence and insertion of T in place thereof; (d) deletion of nucleotide 226 of the coding sequence and insertion of T in place thereof; and (e) deletion of nucleotide 313 of the coding sequence and insertion of A in place thereof; and combinations thereof The second of the pair of primers is preferably located entirely upstream or entirely downstream of the selected mutation or mutations. In one kit, a first said primer spans mutation (a) and further comprising a third primer which is sufficiently complementary to the nucleotide sequence identified as SEQ ID NO:11 to prime amplification of a nucleic acid molecule containing SEQ ID NO:11. In another (or the same kit), a first said primer is sufficiently complementary to the inserted sequence of mutation (b) to prime amplification of a nucleic acid molecule containing mutation (b) and further comprising a third primer which is sufficiently complementary to the sequence corresponding to the 7 nucleotide deletion of mutation (b) to prime amplification of a nucleic acid molecule containing the 7 nucleotide deletion of mutation (b). In another (or the same kit), a first said primer spans mutation (c) and further comprising a third primer which is sufficiently complementary to the sequence spanning the corresponding region lacking mutation (c) to prime amplification of a nucleic acid molecule lacking mutation (c). In another (or the same kit), a first said primer spans mutation (d) and further comprising a third primer which is sufficiently complementary to the sequence spanning the corresponding region lacking mutation (d) to prime amplification of a nucleic acid molecule lacking mutation (d). In another (or the same kit), a first said primer spans mutation (e) and further comprising a third primer which is sufficiently complementary to the sequence spanning the corresponding region lacking mutation (e) to prime amplification of a nucleic acid molecule lacking mutation (e).

The invention includes a purified protein having biological activity of myostatin, and having an amino acid sequence identified as SEQ ID NO:2, or a conservatively substituted variant thereof The invention includes a purified bovine protein having biological activity of myostatin or a purified human protein having biological activity of myostatin.

The invention includes an isolated nucleic acid molecule encoding a foregoing protein. Particularly, the invention includes an isolated nucleic acid molecule comprising a DNA molecule having the nucleotide sequence identified as SEQ ID NO:1 or SEQ ID NO:3 or SEQ ID NO:7 or which varies from the sequence due to the degeneracy of the genetic code, or a nucleic acid strand capable of hybridizing with at least one said nucleic acid molecule under stringent hybridization conditions.

The invention includes isolated mRNA transcribed from DNA having a sequence which corresponds to a nucleic acid molecule of the invention.

The invention includes isolated DNA in a recombinant cloning vector and a microbial cell containing and expressing heterologous DNA of the invention.

The invention includes a transfected cell line which expresses a protein of the invention.

The invention includes a process for producing a protein of the invention, including preparing a DNA fragment including a nucleotide sequence which encodes the protein; incorporating the DNA fragment into an expression vector to obtain a recombinant DNA molecule which includes the DNA fragment and is capable of undergoing replication; transforming a host cell with the recombinant DNA molecule to produce a transformant which can express the protein; culturing the transformant to produce the protein; and recovering the protein from resulting cultured mixture.

The invention includes a method of inhibiting myostatin so as to induce increased muscle mass in a mammal, comprising administering an effective amount of an antibody to myostatin to the mammal.

The invention includes a method of increasing muscle mass in a mammal, by raising an autoantibody to the myostatin the in the mammal. Raising the autoantibody can include administering a protein having myostatin activity to the mammal.

The invention includes a method of increasing muscle mass in a mammal including administering to the mammal an effective amount of an antisense nucleic acid or oligonucleotide substantially complementary to at least a portion of the sequence identified as SEQ ID NO:1 or SEQ ID NO:5, or SEQ ID NO:7. The portion can be at least 5 nucleotide bases in length or longer. The mammal can be a bovine and the sequence can be that identified as SEQ ID NO:1.

The invention includes a method of inhibiting production of myostatin in a mammal in need thereof, including administering to the mammal an effective amount of an antibody to the myostatin.

The invention includes a probe containing a nucleic acid molecule sufficiently complementary with a sequence identified as SEQ ID NO:1, or its complement, so as to bind thereto under stringent conditions. The probe can be a sequence which is between about 8 and about 1195 nucleotides in length.

The invention includes a primer composition useful for detection of the presence of DNA encoding myostatin in cattle. The composition can include a nucleic acid primer substantially complementary to a nucleic acid sequence encoding a bovine myostatin. The nucleic acid sequence can be that identified as SEQ ID NO:1.

The invention includes a method for identifying a nucleotide sequence of a mutant gene encoding a myostatin protein of a mammal displaying muscular hyperplasia. The method includes obtaining a sample of material containing DNA from the mammal and probing the sample using a nucleic acid probe based on a nucleotide sequence of a known gene encoding myostatin in order to identify nucleotide sequence of the mutant gene. In a particular approach, the probe is based on a nucleotide sequence identified as SEQ ID NO:1, SEQ ID NO:5 or SEQ ID NO:7. Preferably, the probe is at least 8 nucleic acids in length. The step of probing the sample can include exposing the DNA to the probe under hybridizing conditions and further comprising isolating hybridized nucleic acid molecules. The method can further include the step of sequencing isolated DNA. The method can include the step of isolating and sequencing a cDNA or mRNA encoding the complete mutant myostatin protein. The method can include a step of isolating and sequencing a functional wild type myostatin from the mammal not displaying muscular hyperplasia.

The method can include comparing the complete coding sequence of the complete mutant myostatin protein with, if the coding sequence for a functional wild type myostatin from such a mammal is previously known, (1) the known sequence, or if the coding sequence for a functional wild type myostatin from such a mammal is previously unknown, (2) the sequence determined according to claim 63 or claim 66, to determine the location of any mutation in the mutant gene.

The invention includes a primer composition useful for the detection of a nucleotide sequence encoding a myostatin containing a first nucleic acid molecule based on a nucleotide sequence located upstream of a mutation determined according to a method of the invention and a second nucleic acid molecule based on a nucleotide sequence located downstream of the mutation.

A probe of the invention can include a nucleic acid molecule based on a nucleotide sequence spanning a mutation determined according to the invention.

The invention includes an antibody to a protein encoded by a nucleotide sequence identified as SEQ ID NO:1, SEQ ID NO:3 or SEQ ID NO:7, or other protein of the present invention.

The invention includes a transgenic bovine having a genome lacking a gene encoding a protein having biological activity of myostatin; a transgenic mouse having a genome containing a gene encoding a human protein having biological activity of myostatin or containing a gene encoding a bovine protein having biological activity of myostatin; a transgenic bovine having a gene encoding a bovine protein having biological activity of myostatin and heterologous nucleotide sequence antisense to the gene. The transgenic bovine can include a gene encoding a nucleic acid sequence having ribozyme activity and in transcriptional association with the nucleotide sequence antisense to the gene.

The invention includes a transgenic mammal, usually non-human, having a phenotype characterized by muscular hyperplasia, said phenotype being conferred by a transgene contained in the somatic and germ cells of the mammal, the transgene encoding a myostatin protein having a dominant negative mutation. The transgenic mammal can be male and the transgene can be located on the Y chromosome. The mammal can be bovine and the transgene can be located to be under the control of a promoter which normally a promoter of a myosin gene.

Another transgenic mammal, usually non-human, of the invention has a phenotype characterized by muscular hyperplasia, in which the phenotype is conferred by a transgene having a sequence antisense to that encoding a myostatin protein of the mammal. The mammal can be a male bovine and the transgene can be located on the Y chromosome. The transgene can further include a sequence which when transcribed obtains an mRNA having ribozyme activity.

A transgenic non-human mammal of the invention having a phenotype characterized by muscular hyperplasia, can have the phenotype inducible and conferred by a myostatin gene flanked by J oxP sides and a Cre transgene under the dependence of an inducible promoter.

A transgenic non-human male mammal of the invention having a phenotype characterized by muscular hyperplasia, can have the phenotype conferred by a myostatin gene flanked by J oxP sides and a Cre transgene located on the Y chromosome.

The invention includes a method for determining whether a sample of mammalian genetic material is capable of a conferring a phenotype characterized by muscular hyperplasia, comprising ascertaining whether the genetic material contains a nucleotide sequence encoding a protein having biological activity of myostatin, wherein the absence of said sequence indicates the presence of muscular hyperplasia in the animal.

### BRIEF DESCRIPTION OF DRAWINGS

In describing particular aspects of the invention, reference is made to the accompanying drawings, in which:

FIG. 1 is a schematic summary of genetic, physical and comparative mapping information around the bovine mh locus. A multi-point lodscore curve obtained for the mh locus with respect to the microsatellite marker map is shown. Markers that were not informative in the pedigree used are shown between brackets; their map position is inferred from published mapping data. Markers and the YACs from which they were isolated are connected by arrows. The RH-map of the relevant section of human HSA2 is shown, with the relative position in cR of the ESTs used. Stippled lines connect microsatellite and Type I markers with their respective positive YACs. YACs showing cross-hybridizing SINE-PCR products are connected by the red boxes.

FIG. 2(a) shows electropherograms obtained by cycle-sequencing the myostatin cDNA sequence from a double-muscled and a conventional animal, showing the nt82Idel(I1) deletion in the double-muscled animal. The primers used to amplify the fragment encompassing the deletion from genomic DNA are spaced apart from the remaining nucleotides.

FIG. 2(b) shows the amino-acid sequence of the murine (top row), bovine normal (middle row) and bovine nt82Idel(I1) (bottom row) allele. The putative site of proteolytic processing is boxed, while the nine conserved cysteines in the carboxy-terminal region are underlined. The differences between the normal and nt82Idel(II) bovine allele are indicated by the double underlining.

FIG. 3 is a schematic representation of the bovine myostatin gene with position and definition of the identified DNA sequence polymorphisms. The “A” (clear) boxes correspond to the untranslated leader and trailer sequences (large diameter), and the intronic sequences (small diameter) respectively. The “B”, “C”, and “D” boxes correspond to the sequences coding for the leader peptide, N-terminal latency-associated peptide and bioactive carboxyterminal domain of the protein respectively. Small “e”, “f” and “g” arrows point towards the positions of the primers used for intron amplification, exon amplification and sequencing and exon sequencing respectively; the corresponding primer sequences are reported in Table 1. The positions of the identified DNA sequence polymorphisms are shown as “h”, “i” or “j” lines on the myostatin gene for silent, conservative and disrupting mutations respectively. Each mutation is connected via an arrow with a box reporting the details of the corresponding DNA sequence and eventually encoded peptide sequence. In each box, the variant sequence is compared with the control Holstein-Friesian sequence and differences are highlighted in color.

FIG. 4 shows the distribution of identified mutations in the various breeds examined. The order of the myostatin mutations correspond to FIG. 3. All analyzed animals were double-muscled except for the two Holstein-Friesian and two Jerseys used as controls (column 1).

### DETAILED DESCRIPTION OF PREFERRED EMBODIMENTS

The method used for isolating genes which cause specific phenotypes is known as positional candidate cloning. It involves: (i) the chromosomal localization of the gene which causes the specific phenotype using genetic markers in a linkage analysis; and (ii) the identification of the gene which causes the specific phenotype amongst the “candidate” genes known to be located in the corresponding region. Most of the time these candidate genes are selected from available mapping information in humans and mice.

The tools required to perform the initial localization (step (i) above) are microsatellite marker maps, which are available for livestock species and are found in the public domain (Bishop et al., 1994; Barendse et al., 1994; Georges et al., 1995; and Kappes, 1997). The tools required for the positional candidate cloning, particularly the YAC libraries, (step (ii) above) are partially available from the public domain. Genomic libraries with large inserts constructed with Yeast Artificial Chromosomes (“YAC”) are available in the public domain for most livestock species including cattle. When cross-referencing the human and mice map, it is necessary to identify the positional candidate, which is available at low resolution but needs to be refined in every specific instance to obtain the appropriate level of high resolution. A number of original strategies are described herein to achieve this latter objective. For general principles of positional candidate cloning, see Collins, 1995 and Georges and Andersson, 1996.

In order to allow for cross-referencing between the bovine and human gene map as part of the positional candidate cloning approach, HSA2q31-32 (map of the long arm of human chromosome 2, cytogenetic bands q31-32) and BTA2q12-22 (map of the arm of bovine chromosome 2, cytogenetic bands q12-22) were integrated on the basis of coincidence bovine YAC's as described below.

Using a previously described experimental [(normal×double-muscled)×double-muscled] backcross population comprising 108 backcross individuals, the mh locus was recently mapped by linkage analysis to the centromeric tip of bovine chromosome 2 (BTA2), at 3.1 centiMorgan proximal from the last marker on the linkage map: TGLA44 (Charlier et al., 1995). It was also known from previous work that pro-α(III) collagen (Col3AI) was located in the same chromosomal region as the mh locus. Col3AI has been mapped to BTA2q12-22 by in situ hybridization (Solinas-Toldo et al., 1995), while a Col3AI RFLP marker was shown to be closely linked to TGLA44 (θ=2%)(Fisher et al., 1996). This identifies the region flanking Col3AI on the human map, i.e. HSA2q31-32, as the likely orthologous human chromosome segment. This assumption is compatible with data from Zoo-FISH experiments (Solinas-Toldo et al., 1995) as well as mapping data of Type I markers on somatic cell hybrids (O'Brien et al., 1993), which establish an evolutionary correspondence between segments of HSA2q and BTA2.

In order to refine the correspondence between the HSA2q31-33 and BTA2q12-22 maps, Comparative Anchored Tagged Sequences or CATS, i.e. primer pairs that would amplify a Sequence Tagged Site or STS from the orthologous gene in different species (Lyons et al., 1996), were developed for a series of genes flanking Col3AI on the human map and for which sequence information was available in more than one mammal. In addition to Col3AI, working CATS were obtained for α2(V) collagen (Col5A2), inositol polyphosphate-1 phosphatase (INPPI), tissue factor pathway inhibitor precursor (TFPI), titin (TTN), n-chimaerin (CHN), glutamate decarboxylase 67 (GADI), Cytotoxic T-lymphocyte-associated protein 4 (CTLA4) and T-cell membrane glycoprotein CD28 (CD28). The corresponding primer sequences are given in Table 1.

TABLE 1

CATS

| | | | | |
|---|---|---|---|---|
| INPP1 | UP: | 5′ CAGCAAAGTCCTTAATGGTAACAAGC 3′ | DN: | 5′ GGGTCACTGAAGAAAACGTCCTG 3′ |
| COL3A1 | UP: | 5′ CCCCATATTATGGAGATGAACCG 3′ | DN: | 5′ AGTTCAGGATGGCAGAATTTCAG 3′ |
| COL5A2 | UP: | 5′ GCAAACTGGGYGGRAGCAAGACC 3′ | DN: | 5′ TTSTTCCTGGGCTTTTATTGAGAC 3′ |
| TFPI | UP: | 5′ AAGCCWGATTTCTGCTTYTTGGAAG 3′ | DN: | 5′ TGCCMAGGCAHCCRCCRTACTTGAA 3′ |
| TTN | UP: | 5′ GGTCGTCCTACACCAGAAG 3′ | DN: | 5′ GGTTGACATTGTCAAGAACAAG 3′ |
| CHN | UP: | 5′ TCTCMAAAGTCGTCTGTGACAATC 3′ | DN: | 5′ TGYTCRTTTTCTTTCAGAGTTGC 3′ |
| GAD1 | UP: | 5′ RCTGGTCCTCTTCACCTCAGAAC 3′ | DN: | 5′ ACATTGTCVGTTCCAAAGCCAAG 3′ |
| CTLA4 | UP: | 5′ AGGTYCGGGTGACDGTGCTKC 3′ | DN: | 5′ TGGRTACATGAGYTCCACCTTGC 3′ |
| CD28 | UP: | 5′ AGCTGCARGTATWCCTACAAYCT 3′, | DN: | 5′ GTYCCRTTGCTCYTCTCRTTGYC 3′ |

Microsatellite markers

| | | | | |
|---|---|---|---|---|
| TGLA44 | UP: | 5′ AACTGTATATTGAGAGCCTACCATG 3′ | DN: | 5′ CACACCTAGCGACTAAACCACCA 3′ |
| BULGE27 | UP: | 5′ CTACCTAACAGAATGATTTTGTAAG 3′ | DN: | 5′ AGTGTTCTTGCCTAGAGAATCCCAG 3′ |
| BULGE23 | UP: | 5′ ACATTCTCTCACCAATATGACATAC 3′ | DN: | 5′ TAAGTCACCATTACATCCTTAGAAC 3′ |
| BM81124 | UP: | 5′ GCTGTAAGAATCTTCATTAAGCACT 3′ | DN: | 5′ CCTGATACATGCTAAGGTGAAAAAC 3″ |
| BULGE28 | UP: | 5′ AGGCATACATCTGGAGAGAAACATG 3′ | DN: | 5′ CAGAGGAGCCTAGCAGGCTACCGTC 3′ |
| BULGE20 | UP: | 5′ CAGCAGGTCTGTTGAAGTGTATCAG 3′ | DN: | 5′ AGTGGTAGCATTCACAGGTAGCCAG 3′ |
| BM3627 | UP: | 5′ CAGTCCATGGCACCATAAAG 3′ | DN: | 5′ TCCGTTAGTACTGGCTAATTGC 3′ |
| ILSTS026 | UP: | 5′ CTGAATTGGCTCCAAAGGCC 3′ | DN: | 5′ AAACAGAAGTCCAGGGCTGC 3′ |
| INRA40 | UP: | 5′ TCAGTCTCCAGGAGAGAAAAC 3′ | DN: | 5′ CTCTGCCCTCGGGGATGATTG 3′ |

Bovine Myostatin primers

| | |
|---|---|
| GDF8.19 | 5′ AATGTATGTTTATATTTACCTGTTCATG 3′ |
| GDF8.11 | 5′ ACAGTGTTTGTGCAAATCCTGAGAC 3′ |
| GDF8.12 | 5′ CAATGCCTAAGTTGGATTCAGGTTG 3′ |
| GDF8.25 | 5′ CTTGCTGTAACCTTCCCAGGACCAG 3′ |
| GDF8.15 | 5′ TCCCATCCAAAGGCTTCAAAATC 3′ |
| GDF8.21 | 5′ ATACTCWAGGCCTAYAGCCTGTGGT 3′ |

Reading from left to right and down the table, the sequences given in Table 1 are identified as SEQ ID NO:12 to SEQ ID NO:53, respectively.

These CATS were used to screen a 6-genome equivalent bovine YAC library by PCR using a three-dimensional pooling strategy as described by Libert et al., 1993. The same YAC library was also screened with all microsatellite markers available for proximal BTA2, i.e. TGLA44, BM81124, BM3627, ILSTS026, INRA40 and TGLA431 (Kappes et al., 1997).

Potential overlap between the YACs obtained with this panel of STS's was explored on the basis of common STS content, as well as cross-hybridization between SINE-PCR product from individual YACs. From this analysis, three independent YAC contigs emerged in the region of interest: (i) contig A containing microsatellites TGLA44, BM81124 and Type I marker INPP1; (ii) contig B containing Col3AI and Col5A2; and (iii) contig C containing microsatellite markers BM3627, iLSTS026 and InRA40, and Type I marker TFPI.

None of the available microsatellites mapped to contig B, therefore this cluster of YACs could not be positioned in cattle with respect to the two other contigs. Available mapping information in the human, however, allowed prediction of contig B's position between contigs A and C. To test this hypothesis, two new microsatellite markers were isolated from contig B, BULGE20 and BULGE28. BULGE20 proved to be polymorphic, allowing for genotyping of the experimental backcross population.

In addition, to increase the informativeness of the markers available for contig A, two new microsatellite markers were developed from this contig: BULGE23 and BULGE27. BULGE23 proved to be polymorphic and was used to type the same pedigree material.

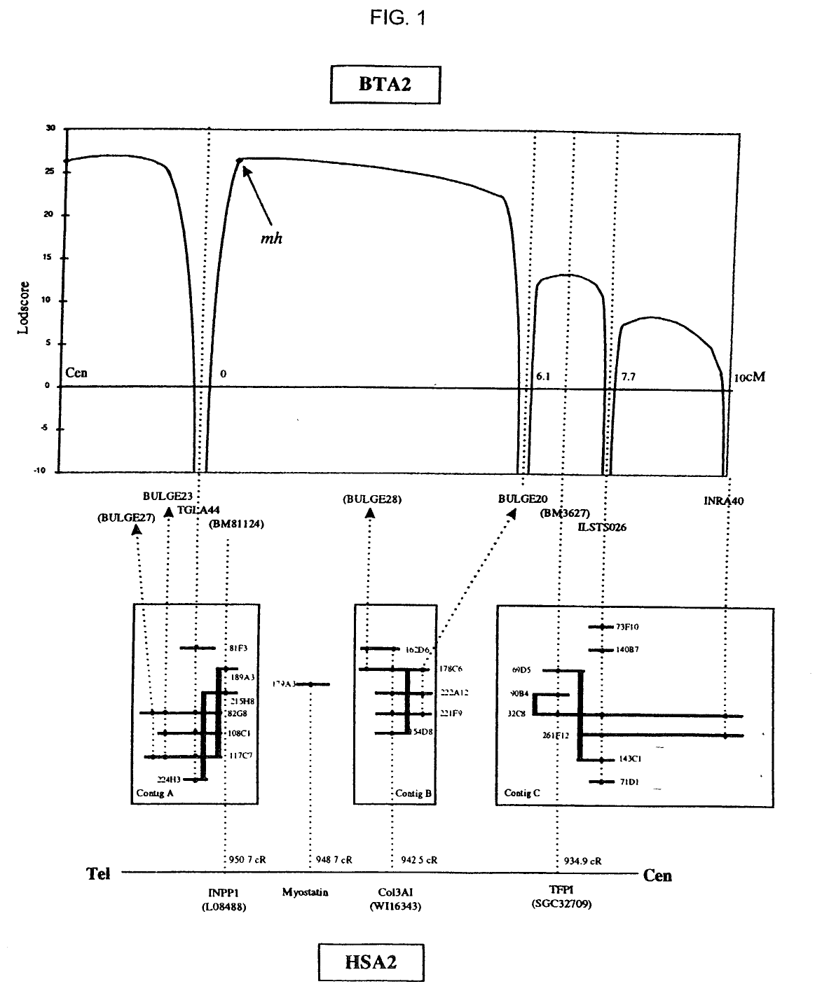

All resulting genotypes were used to construct a linkage map using the ILINK program (Lathrop and Lalouel, 1984). The following most likely order and sex-averaged recombination rates between adjacent markers was obtained: [TGLA44-(0%)-BULG23]-(6,1%)-BULG20-(1,6%)-ILSTS026-(2.3%)-INRA40-(7,1%)-TGLA431. The position of BULGE20 between TGLA44 and ILSTS026 confirmed the anticipated order of the three contigs. FIG. 1 summarizes the resulting mapping information.

A multi point linkage analysis was undertaken using LINKMAP, to position the mh locus with respect to the new marker map. Linkage analysis was performed under a simple recessive model, assuming full penetrance for mh/mh individuals and zero penetrance for the two other genotypes. The LOD score curve shown in FIG. 1 was obtained, placing the mh locus in the TGLA44-BULGE20 interval with an associated maximum LOD score of 26.4. Three backcross individuals were shown to recombine with the BULGE20 and distal markers, but not with TGLA44 and BULGE23, therefore placing the mh locus proximal from this marker. One individual, was shown to recombine with TGLA44 and BULGE23, but not with the more distal markers, therefore positioning the mh locus distal from TGLA44 and BULGE23. Given the relative position of these microsatellite markers with respect to INPPI and Col3AI as deduced from the integration of the human and bovine map, these results indicated that the mh gene is likely located in a chromosome segment bounded by INPPI and Col3AI.

Recently, McPherron et al. (1997) demonstrated that mice homozygous for a knock-out deletion of GDF-8 or myostatin, were characterized by a generalized increase in skeletal muscle mass. Using the published 2676 bp murine myostatin cDNA sequence (GenBank accession number U84005), a Tentative Human Consensus (THC) cluster in the Unigene database was identified which represented three cDNA clones (221299, 300367, 308202) and six EST (Expressed Sequence Tag) sequences (H92027, H92028, N80248, N95327, W07375, W24782). The corresponding THC covered 1296 bp of the human myostatin gene, showing an homology of 78.1% with the murine sequence when averaged over the entire sequence, and 91.1% when considering only the translated parts of the human and murine genes (566 bp). This THC therefore very likely corresponds to the human orthologue of the murine myostatin gene. Primers (5′-GGCCCAACTATGGATATATTTG-3′ (SEQ ID NO:9) and 5′-GGTCCTGGGAAGGTTACAGCA-3′ (SEQ ID NO:10)) were thus prepared to amplify a 272 bp fragment from the second exon of human myostatin and used to genotype the whole-genome Genebridge-4 radiation hybrid panel (Walter et al, 1994). The RHMapper program (Slonim et al., unpublished) was used to position the myostatin gene with respect to the Whitehead/MIT framework radiation hybrid map, placing it at position 948.7 cR of the HSA2 map with an associated lodscore >3. Closer examination of the myostatin segregation vector and its confrontation with the vectors from all markers located in that region (Data Release 11.9, May 1997) showed it to be identical to EST SGC38239 placed on the Whitehead/MIT radiation hybrid map (Hudson et al., 1995) at position 946.8 cR of HSA2. This places the human myostatin gene on the RH-map in the interval between Col3AI (EST WI16343-942.5 cR) and INPPI (EST L08488-950.2 to 951.2 cR)(Figure 1). Myostatin therefore appeared as a very strong positional candidate for the mh gene.

To test the potential involvement of myostatin in the determinism of double-muscling in cattle, primer pairs were designed based on the available mouse and human myostatin sequence, with the objective to amplify the entire coding sequence from bovine cDNA using PCR (Polymerase Chain Reaction). Whenever possible, primers were therefore positioned in portions of the myostatin sequence showing 100% homology between mouse and human. Two primer pairs were identified that amplified what was predicted to represent 98.4% of the bovine coding sequence plus 74 bp of 3′ untranslated sequence, in two overlapping DNA fragments, respectively 660 (primers GDF8.19-GDF8.12) and 724 bp (primers GDF8.11-GDF8.21) long. The expected DNA products were successfully amplified from cDNA generated from skeletal muscle of both a normal (homozygous +/+) (SEQ ID NO:1) and a double-muscled (homozygous mh/mh) (SEQ ID NO:3) animal, and cycle-sequenced on both strands.

The nucleotide sequence corresponding to the normal allele presented 88.9% identity with the mouse myostatin sequence (SEQ ID NO:5) over a 1067 bp overlap, and contained the expected open reading frame encoding a protein (SEQ ID NO:2) showing 92.9% identity in a 354 amino-acid overlap with mouse myostatin (SEQ ID NO:6). As expected for a member of the TGFβ superfamily, the bovine myostatin gene is characterized by a proteolytic processing site thought to mediate cleavage of the bioactive carboxy-terminal domain from the longer N-terminal fragment, and by nine cysteine residues separated by a characteristic spacing and suspected to be involved in intra- and inter-molecular disulfide bridges (McPherron and Lee, 1996).

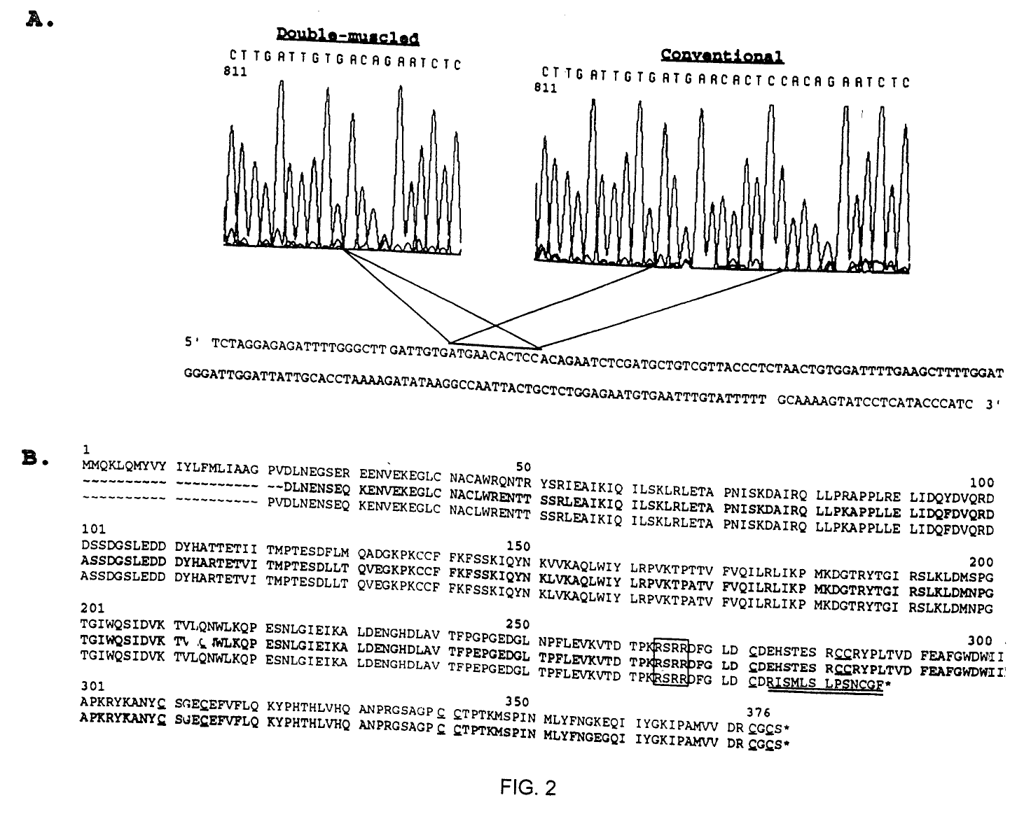

The nucleotide sequence obtained from the mh allele was identical to the normal allele over its entire length, except for an 11 bp deletion involving nucleotides 821 to 831 (counting from the initiation codon). This frame shifting deletion, occurring after the first cysteine residue of the carboxy-terminal domain, drastically disrupts the downstream amino-acid sequence and reveals a premature stop-codon after 13 amino acids, see FIG. 2. This mutation disrupts the bioactive part of the molecule and is therefore very likely to be the cause of the recessive double-muscling phenotype. Following conventional nomenclature, this mutation will be referred to as nt821(del11).

To further strengthen the assumption of the causality of this mutation, primer pairs flanking the deletion (FIG. 2) were prepared and the corresponding DNA segment from all animals from the experimental backcross population amplified. PCR was performed in the presence of dCTP³² in order to radioactively label the amplification product. Amplification products were separated on denaturing polyacrylamide gels and detected by autoradiography. A 188 bp product would be expected for the normal allele and a 177 bp product for the nt821(del11) allele. Correlation between phenotype and genotype was matched for the entire pedigree. All ten BBCB double-muscled sires were found to be homozygous for the nt821(del11) mutation, all 41 F1 females were heterozygous, while 53 double-muscled offspring were homozygous for the mutation, the remaining 55 conventional animals were heterozygous.

To examine the distribution of the nt821(del11) mutation in different conventional and double-muscled breeds, a cohort of 25 normal individuals was genotyped representing two dairy breeds (Holstein-Friesian, Red-and -White) and a cohort of 52 double-muscled animals representing four breeds (BBCB, Asturiana, Maine-Anjou and Piémontese). The results are summarized in Table 2. All dairy animals were homozygous normal except for one Red-and-White bull shown to be heterozygous. The occurrence of a small fraction of individuals carrying the mutation in dairy cattle is not unexpected as the phenotype is occasionally described in this breed. In BBCB and Asturiana, all double-muscled animals were homozygous for the nt821(del11) deletion, pointing towards allelic homogeneity in these two breeds. Double-muscled Maine-Anjou and Piémontese animals were homozygous “normal”, i.e. they did not show the nt821(del11) deletion but a distinct cysteine to tyrosine substitution (C313Y) in double-muscled Piédmontese animals identified by others (Kambadur et al., 1997) was discovered.

TABLE 2

| | | Genotype | | |
|---|---|---|---|---|
| | | | | nt821(del11)/ |
| Breed | Phenotype | +/+ | +/nt821(del11) | nt821(del11) |
| Belgian Blue | DM | | | 29 |
| Asturiana | DM | | | 10 |
| Piémontese | DM | 8 | | |
| Maine-Anjou | DM | 4 | | |
| Holstein- | Normal | 13 | | |
| Friesian | | | | |
| Red-and-White | Normal | 12 | 1 | |

The entire coding sequence was also determined for the myostatin gene in double-muscled individuals from ten European cattle breeds and a series of mutations that disrupt myostatin function were identified.

The coding sequence of four control Holstein-Friesian and Jersey individuals was identical to the previously described wild-type allele (Grobet et al., 1997), further indicating that it was the genuine myostatin coding sequence being amplified, and not a non-functional pseudogene.

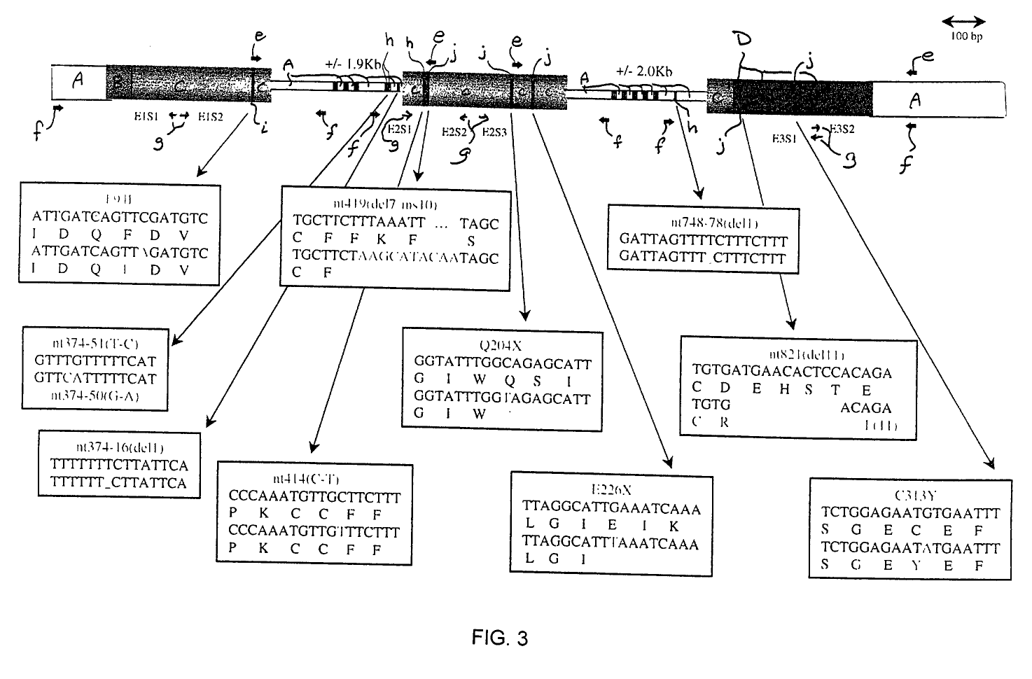

Amongst the 32 double-muscled animals, seven DNA sequence variants within the coding region were found, as summarized in FIG. 3.

In addition to the nt821(del11) mutation in the third exon, described above, four new mutations that would be expected to disrupt the myostatin function were found. An insertion/deletion at position 419 counting from the initiation codon, replacing 7 base pairs with an apparently unrelated stretch of 10 base pairs, reveals a premature stop codon in the N-terminal latency-associated peptide at amino-acid position 140. This mutation is referred to as nt419(del7-ins10). Two base pair substitutions in the second exon, a C→T transition at nucleotide position 610 and a G→T transversion at nucleotide position 676, each yield a premature stop codon in the same N-terminal latency-associated peptide at amino-acid positions 204 and 226 respectively. These mutations are called Q204X and E226X respectively. Finally, a G→A transition at nucleotide position 938 results in the substitution of a cysteine by a tyrosine. This mutation is referred to as C313Y. This cysteine is the fifth of nine highly conserved cysteine residues typical of the members of the TGF-β superfamily and shared in particular by TGF-β1, -β2 and -β3, and inhibin-βA and -βB (McPherron & Lee, 1996). It is thought to be involved in an intramolecular disulfide bridge stabilizing the three-dimensional conformation of the bioactive carboxyterminal peptide. Its substitution is therefore likely to affect the structure and function of the protein. This C313Y has recently also been described by Kambadur et al (1997).

A conservative phenylalanine to leucine substitution was also found at amino-acid position 94 in the first exon, due to a C→A transversion at nucleotide position 282 of the myostatin gene. Given the conservative nature of the amino-acid substitution, its location in the less conserved N-terminal latency-associated peptide, and as this mutation was observed at the homozygous condition in animals that were not showing any sign of exceptional muscular development, this mutation probably does not interfere drastically with the myostatic function of the encoded protein, if at all. This mutation is referred to as F94L. The murine protein is characterized by a tyrosine at the corresponding amino-acid position.

Also identified was a silent C→T transition at the third position of the 138th cytosine codon in the second exon, referred to as nt414(C→T).

In addition to these DNA sequence polymorphisms detected in the coding region of the myostatin gene, also found were four DNA sequence variants in intronic sequences which are probably neutral polymorphisms and which have been assigned the following symbols: nt374-51(T-C), nt374-50(G-A), nt374-16(del1) in intron 1, and nt748-78(del1) in intron 2 (FIG. 3).

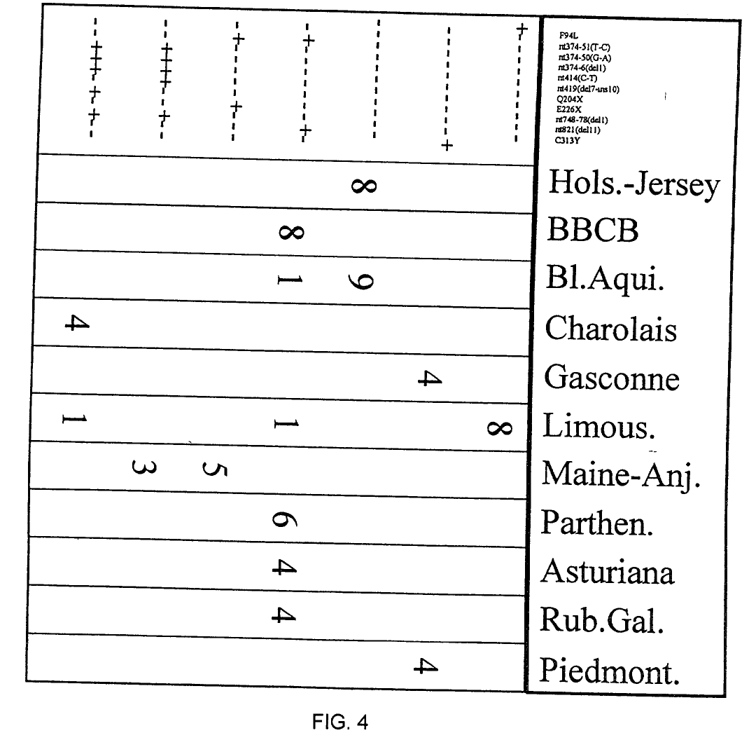

FIG. 4 shows the observed distribution of mutations in the analysed sample sorted by breed. For the majority of the studied breeds, the analyzed double-muscled animals were homozygous for one of the five described mutations expected to disrupt the myostatin function or compound heterozygotes for two of these mutations. This is compatible with the hypothesis that the double-muscled condition has a recessive mode of inheritance in all these breeds.

Only in Limousin and Blonde d'Aquitaine was there no clear evidence for the role of myostatin loss-of-function mutations in the determinism of the observed muscular hypertrophy. Most Limousin animals were homozygous for the conservative F94L substitution which is unlikely to cause the muscular hypertrophy characterizing these animals, as discussed above. One Limousin animal proved to be heterozygous for this mutation, the other allele being the “wild-type” one. All Blonde d'Aquitaine animals were homozygous “wild-type”. These data indicate either that the myostatin gene is possibly not involved in the double-muscled condition characterizing these two breeds, or that there are additional myostatin mutations outside of the coding region. The double-muscling condition is often considered to be less pronounced in Limousin animals compared to other breeds.

The data indicate that some mutations, such as the nt821del(11) and C313Y, are shared by several breeds which points towards gene migration between the corresponding populations, while others seems to be confined to specific breeds. Moreover, while some breeds (the Belgian Blue breed in particular) seem to be essentially genetically homogeneous others show clear evidence for allelic heterogeneity (e.g. Maine-Anjou).

The observation of allelic heterogeneity contradicts with the classical view that a single mh mutation spread through the European continent in the beginning of the 19th century with the dissemination of the Shorthorn breed from the British Isles (Ménissier, 1982). Two of the mutations at least are shared by more than one breed, indicating some degree of gene migration but definitely not from a single origin.

In mice, and in addition to the in vitro generated myostatin knock-out mice (McPherron & Lee, 1997), the compact mutation could be due to a naturally occurring mutation at the myostatin gene. The compact locus has been mapped to the D1Mit375-D1Mit21 interval on mouse chromosome 1 known to be orthologous to HSA2q31-32 and BTA2q12-22 (Varga et al., 1997).

From an applied point of view, the characterisation of a panel of mutations in the myostatin gene associated with double-muscling contributes to the establishment of a diagnostic screening system allowing for marker assisted selection for or against this condition in cattle.

### EXAMPLE 1

### Genetic and physical mapping

Integration of the HSA2q31-32 and BTA2q12-22 maps was done by using coincident YAC's and the mh locus was positioned in the interval flanked by Col3AI and INPP1 as follows. Genetic mapping was performed using a previously described (Holstein-Friesian×Belgian Blue)×Belgian Blue experimental backcross population counting 108 informative individuals (Charlier et al., 1995). Microsatellite genotyping was performed according to standard procedures (Georges et al., 1995), using the primer sequences reported in Table 1. Linkage analyses were performed with the MLINK, ILINK and LINKMAP programs of the LINKAGE (version 5.1) and FASTLINK (2.3P version, June 1995) packages (Lathrop & Lalouel, 1984; Cottingham et al., 1993). The YAC library was screened by PCR using a three dimensional pooling scheme as described in Libert et al., 1993. The primer pairs corresponding to the CATS used to screen the library are reported in Table 1. Cross-hybridisation between SINE-PCR products of individual YACs was performed according to Hunter et al. (1996), using primers reported in Lenstra et al. (1993). Microsatellites were isolated from YACs according to Cornelis et al. (1992).

### EXAMPLE 2

### Mapping of the human myostatin gene on the Genebridge-4-panel

DNA from the Genebridge-4 panel (Walter et al., 1994) was purchased from Research Genetics (Huntsville, Alabama), and genotyped by PCR using standard procedures and the following human myostatin primer pair (5′-GGCCCAACTATGGATATATTTG-3′ and 5′-GGTCCTGGGAAGGTTACAGCA-3′). Mapping was performed via the WWW server of the Whitehead Institute/MIT Center for Genome Research using their RH-mapper program (Slonim, D.; Stein, L.; Kruglyak, L.; Lander, E., unpublished) to position the markers with respect to the framework map. Segregation vectors of the query markers were compared with the vectors from all markers in the region of interest in the complete Data Release 11.9 (May 1997) to obtain a more precise position. This positions myostatin in the INPPI-Col3AI on the human map with LOD score superior to 3.

### EXAMPLE 3

### RT-PCR

To clone the bovine myostatin orthologue a strategy based on RT-PCR amplification from skeletal muscle cDNA was chosen. Total RNA was extracted from skeletal muscle (Triceps brachialis) according to Chirgwin et al. (1979). RT-PCR was performed using the Gene-Amp RNA PCR Kit (Perkin-Elmer) and the primers reported in Table 1. The PCR products were purified using QiaQuick PCR Purification kit (Qiagen) and sequenced using Dye terminator Cycle Sequencing Ready Reaction (Perkin-Elmer) and an ABI373 automatic sequencer, using the primers reported in Table 2.

### EXAMPLE 4

### Diagnosis of the nt821(del11) deletion

To diagnose the nt821 (del11) the following primer sequences were designed flanking the nt821(del11) deletion: 5′-TCTAGGAGAGATTTTGGGCTT-3′ (SEQ ID NO:53) and 5-GATGGGTATGAGGATACTTTTGC-3′ (SEQ ID NO:52). These primers amplify a 188 bp fragments from normal individuals and a 177 bp fragment from double-muscled individuals. Heterozygous individuals show the two amplification products. These amplification products can be detected using a variety of methods. In this example the PCR product was labelled by incorporation of dCTP³², separated on a denaturing acrylamide gel and revealed by autoradiography. Other approaches that could be used to distinguish the three different genotypes are known to those skilled in the art and would include separation in agarose gels and visualization with ethidium bromide, direct sequencing, TaqMan assays, hybridization with allele specific oligonucleotides, reverse dot-blot, RFLP analysis and several others. The specificity of the test is linked to the detected mutation and not to the primers used in the detection method. That means that other primers can easily be designed based on said bovine myostatin sequence that would fulfill the same requirements.

### EXAMPLE 5

### Determination of mutations in other breeds

A total of 32 animals with extreme muscular development were sampled from ten European beef cattle breeds in which double-muscled animals are known to occur at high to moderate frequency: (i) Belgium: Belgian Blue (4), (ii) France: Blonde d'Aquitaine (5), Charolais (2), Gasconne (2), Limousin (5), Maine-Anjou (4), Parthenaise (3), (iii) Spain: Asturiana (2), Rubia Gallega (2), (iv) Italy: Piedmontese (2). The determination of the double-muscled phenotype of the sampled animals was performed visually by experienced observers. Four animals with conventional phenotype sampled from the Holstein-Friesian (2) and Jersey (2) dairy populations were analysed as controls.

In order to facilitate the study of the myostatin coding sequence from genomic DNA, the sequences of the exon-intron boundaries of the bovine gene were determined. In mice, the myostatin gene is known to be interrupted by two introns, respectively ≈1.5 and 2.4 Kb long (McPherron & Lee, 1997). Two primer pairs were thus designed, respectively, in bovine exons 1 and 2, and exons 2 and 3, that were predicted to flank the two introns, assuming conservation of gene organisation between mouse and cattle (FIG. 3 and Table 3). Using these primer sets, two PCR products respectively 2 Kb and 3.5 Kb long were generated from a YAC clone (179A3) containing the bovine myostatin gene (Grobet et al., 1997). The PCR products were purified using QiaQuick PCR Purification kit (Qiagen) and partially sequenced using Dye terminator Cycle Sequencing Ready Reaction (Perkin-Elmer) and an ABI373 automatic sequencer. Alignment with the bovine cDNA sequence identified the four predicted exon-intron boundaries. The nucleotide sequence corresponding to bovine genomic DNA is identified as SEQ ID NO:54.

TABLE 3
Primers used for PCR amplification and cycle sequencing.

| | | | |
|---|---|---|---|
| Intron1-5′ | 5′-GAAGACGATGACTACCACGCCAGGACG-3′ | Intron1-3′ | 5′-CTAGTTTATTGTATTGTATCTTAGAGC-3′ |
| Intron2-5′ | 5′-AGACTCCTACAACAGTGTTTGT-3′ | Intron2-3′ | 5′-ATACTCWAGGCCTAYAGCCTGTGGT-3′ |
| Exon1-5′ | 5′-ATTCACTGGTGTGGCAAGTTGTCTCTCAG Exon1-3′ | 5′-CCCTCCTCCTTACATACAAGCCAGCAG-3′ | |
| | A-3 | | |
| Exon2-5′ | 5′-GTTCATAGATTGATATGGAGGTGTTCG-3′ | Exon2-3 | 5′-ATAAGCACAGGAAACTGGTAGTTATT-3′ |
| Exon3-5′ | 5′-GAAATGTGACATAAGCAAAATGATTAG-3′ | Exon3-3′ | 5′-ATACTCWAGGCCTAYAGCCTGTGGT-3′ |
| Exon1-Seq1 | 5′-TTGAGGATGTAGTGTTTTCC-3′ | Exon1-Seq2 | 5′-GCCATAAAAATCCAAATCCTCAG-3′ |
| Exon2-Seq1 | 5′-CATTTATAGCTGATCTTCTAACGCAAG-3′ | Exon2-Seq2 | 3′-TGTCGCAGGAGTCTTGACAGGCCTCAG-3′ |
| Exon2-Seq3 | 5′-GTACAAGGTATACTGGAATCCGATCTC-3′ | | |
| Exon3-Seq1 | 5′-AGCAGGGGCCGGCTGAACCTCTGGG-3′ | Exon3-Seq2 | 5′-CCCCAGAGGTTCAGCCGGCCCCTGC-3′ |

Based on the available exonic and intronic sequences of the bovine myostatin gene, three primer pairs that jointly allow for convenient amplification of the entire coding sequence from genomic DNA were designed. The position of the corresponding primers is shown in FIG. 3, and the corresponding sequences are reported in Table 3.

After PCR amplification of the entire coding sequence from genomic DNA in the three described fragments, these were purified using QiaQuick PCR Purification kit (Qiagen) and sequenced using Dye terminator Cycle Sequencing Ready Reaction (Perkin-Elmer) and an ABI373 automatic sequencer, using the primers used for amplification as well as a series of nested primers (FIG. 3 and Table 3). Chromat files produced with the ABI373 sequencer were analysed with the Polyphred application (D. Nickerson, personal communication), which is part of a series of sequence analysis programs including Phred (Ewing, B. & Green, P. (1992), unpublished), Phrap (Green, P. (1994), unpublished) and Consed (Gordon, D. (1995), unpublished), but any suitable sequencing programme would do, as known to a person skilled in the art.

Monoclonal antibodies (Mab's) specific for myostatin are useful. In the case of the bovine protein having the amino acid sequence identified as SEQ ID NO:2, for example, antibodies can be used for diagnostic purposes such as for determining myostatin protein levels in muscle tissue. To produce these antibodies, purified myostatin is prepared. The myostatin can be produced in bacterial cells as a fusion protein with glutathione-S-transferase using the vector pGEX2 (Pharmacia). This permits purification of the fusion protein by GSH affinity chromatography. In another approach, myostatin is expressed as a fusion protein with the bacterial maltose binding domain. The fusion protein is thus recovered from bacterial extracts by passing the extract over an amylose resin column followed by elution of the fusion protein with maltose. For this fusion construct, the vector pMa1C2, commercially available from New England Biolabs, can be used. The preparation of a second fusion protein is also useful in the preliminary screening of MAb's.

The generation of hybridomas expressing monoclonal antibodies recognizing myostatin protein is carried out as follows: BALB/c mice are injected intraperitoneally with protein/adjuvant three times at one-month intervals, followed by a final injection into the tail vein shortly prior to cell fusion. Spleen cells are harvested and fused with NS-1 myeloma cells (American Type Culture Collection, Rockville, Md.) using polyethylene glycol 4000 according to standard protocols (Kennett, 1979; Mirski, 1989). The cell fusion process is carried out as described in more detail below.

The fused cells are plated into 96-well plates with peritoneal exudate cells and irradiated spleen cells from BALB/Ccmice as feeder layers and selection with hypoxanthine, aminopterin, and thymidine (HAT medium) is performed.

An ELISA assay is used as an initial screening procedure. 1-10 μg of purified myostatin (cleaved from the fusion protein) in PBS is used to coat individual wells, and 50-100 μl per well of hybridoma supernatants is incubated. Horseradish peroxidase-conjugated anti-mouse antibodies are used for the colorimetric assay.

Positive hybridomas are cloned by limiting-dilution and grown to large-scale for freezing and antibody production. Various positive hybridomas are selected for usefulness in western blotting and immunohistochemistry, as well as for cross reactivity with myostatin proteins from different species, for example the mouse and human proteins.

Alternatively, active immunization by the generation of an endogenous antibody by direct exposure of the host animal to small amounts of antigen can be carried out. Active immunization involves the injection of minute quantities of antigen (g) which probably will not induce a physiological response and will be degraded rapidly. Antigen will only need to be administered as prime and boost immunizations in much the same manner as techniques used to confer disease resistance (Pell et al., 1997).

Antisense nucleic acids or oligonucleotides (RNA or preferably DNA) can be used to inhibit myostatin production in order to increase muscle mass of an animal. Antisense oligonucleotides, typically 15 to 20 bases long, bind to the sense mRNA or pre mRNA region coding for the protein of interest, which can inhibit translation of the bound mRNA to protein. The cDNA sequence encoding myostatin can thus be used to design a series of oligonucleotides which together span a large portion, or even the entire cDNA sequence. These oligonucleotides can be tested to determine which provides the greatest inhibitory effect on the expression of the protein (Stewart, 1996). The most suitable mRNA target sites include 5′- and 3′-untranslated regions as well as the initiation codon. Other regions might be found to be more or less effective. Alternatively, an antisense nucleic acid or oligonucleotide may bind to myostatin coding or regulatory sequences.

Rather than reducing myostatin activity by inhibiting myostatin gene expression at the nucleic acid level, activity of the myostatin protein may be directly inhibited by binding to an agent, such as, for example, a suitable small molecule or a monoclonal antibody.

It will of course be understood, without the intention of being limited thereby, that a variety of substitutions of amino acids is possible while preserving the structure responsible for myostatin activity of the proteins disclosed herein. Conservative substitutions are described in the patent literature, as for example, in U.S. Pat. No. 5,264,558. It is thus expected, for example, that interchange among non-polar aliphatic neutral amino acids, glycine, alanine, proline, valine and isoleucine, would be possible. Likewise, substitutions among the polar aliphatic neutral amino acids, serine, threonine, methionine, asparagine and glutamine could possibly be made. Substitutions among the charged acidic amino acids, aspartic acid and glutamic acid, could probably be made, as could substitutions among the charged basic amino acids, lysine and arginine. Substitutions among the aromatic amino acids, including phenylalanine, histidine, tryptophan and tyrosine would also likely be possible. These sorts of substitutions and interchanges are well known to those skilled in the art. Other substitutions might well be possible. Of course, it would also be expected that the greater the percentage of homology, i.e., sequence similarity, of a variant protein with a naturally occurring protein, the greater the retention of metabolic activity. Of course, as protein variants having the activity of myostatin as described herein are intended to be within the scope of this invention, so are nucleic acids encoding such variants.

A further advantage may be obtained through chimeric forms of the protein, as known in the art. A DNA sequence encoding the entire protein, or a portion of the protein, could thus be linked, for example, with a sequence coding for the C-terminal portion of E. coli β-galactosidase to produce a fusion protein. An expression system for human respiratory syncytial virus glycoproteins F and G is described in U.S. Pat. No. 5,288,630 issued Feb. 22, 1994 and references cited therein, for example.

A recombinant expression vector of the invention can be a plasmid, as described above. The recombinant expression vector of the invention further can be a virus, or portion thereof, which allows for expression of a nucleic acid introduced into the viral nucleic acid. For example, replication defective retroviruses, adenoviruses and adeno-associated viruses can be used.

The recombinant expression vectors of the invention can be used to make a transformant host cell including the recombinant expression vector. The term “transformant host cell” is intended to include prokaryotic and eukaryotic cells which have been transformed or transfected with a recombinant expression vector of the invention. The terms “transformed with”, “transfected with”, “transformation” and “transfection” are intended to encompass introduction of nucleic acid (e.g. a vector) into a cell by one of many possible techniques known in the art. Prokaryotic cells can be transformed with nucleic acid by, for example, electroporation or calcium-chloride mediated transformation. Nucleic acid can be introduced into mammalian cells via conventional techniques such as calcium phosphate or calcium chloride coprecipitation, DEAE-dextran-mediated transfection, lipofection, electroporation or microinjection. Suitable methods for transforming and transfecting host cells are known (Sambrook, 1989).

The number of host cells transformed with a recombinant expression vector of the invention by techniques such as those described above will depend upon the type of recombinant expression vector used and the type of transformation technique used. Plasmid vectors introduced into mammalian cells are integrated into host cell DNA at only a low frequency. In order to identify these integrants, a gene that contains a selectable marker (e.g. resistance to antibiotics) is generally introduced into the host cells along with the gene of interest. Preferred selectable markers include those which confer resistance to certain drugs, such as G418 and hygromycin. Selectable markers can be introduced on a separate plasmid from the nucleic acid of interest or, preferably, are introduced on the same plasmid. Host cells transformed with one or more recombinant expression vectors containing a nucleic acid of the invention and a gene for a selectable marker can be identified by selecting for cells using the selectable marker. For example, if the selectable marker encodes a gene conferring neomycin resistance (such as pRc/CMV), transformant cells can be selected with G418. Cells that have incorporated the selectable marker gene will survive, while the other cells die.

Nucleic acids which encode myostatin proteins can be used to generate transgenic animals. A transgenic animal (e.g., a mouse) is an animal having cells that contain a transgene, which transgene is introduced into the animal or an ancestor of the animal at a prenatal, e.g., an embryonic stage. A transgene is a DNA which is integrated into the genome of a cell from which a transgenic animal develops. In one embodiment, a bovine cDNA, comprising the nucleotide sequence shown in SEQ ID NO:1, or an appropriate variant or subsequence thereof, can be used to generate transgenic animals that contain cells which express bovine myostatin. Likewise, variants such as mutant genes (e.g. SEQ ID NO:3) can be used to generate transgenic animals. This could equally well be done with the human myostatin protein and variants thereof “Knock out” animals, as described above, can also be generated. Methods for generating transgenic animals, particularly animals such as mice, have become conventional in the art are described, for example, in U.S. Pat. Nos. 4,736,866 and 4,870,009. In a preferred embodiment, plasmids containing recombinant molecules of the invention are microinjected into mouse embryos. In particular, the plasmids are microinjected into the male pronuclei of fertilized one-cell mouse eggs; the injected eggs are transferred to pseudo-pregnant foster females; and, the eggs in the foster females are allowed to develop to term. (Hogan, 1986). Alternatively, an embryonal stem cell line can be transfected with an expression vector comprising nucleic acid encoding a myostatin protein, and cells containing the nucleic acid can be used to form aggregation chimeras with embryos from a suitable recipient mouse strain. The chimeric embryos can then be implanted into a suitable pseudopregnant female mouse of the appropriate strain and the embryo brought to term. Progeny harboring the transfected DNA in their germ cells can be used to breed uniformly transgenic mice.

Such animals could be used to determine whether a sequence related to an intact myostatin gene retains biological activity of myostatin. Thus, for example, mice in which the murine myostatin gene has been knocked out and containing the nucleic acid sequence identified as SEQ ID NO:1 could be generated along with animals containing the nucleic acid sequence identified as SEQ ID NO:3. The animals could be examined for display of muscular hyperplasia, especially in comparison with knockout mice, which are known to display such. In this way it can be shown that the protein encoded by SEQ ID NO:3 lacks myostatin activity within the context of this invention while the protein encoded by the nucleic acid sequence identified as SEQ ID NO:1 possesses biological activity of myostatin.

In such experiments, muscle cells would be particularly targeted for myostatin (and variants) transgene incorporation by use of tissue specific enhancers operatively linked to the encoding gene. For example, promoters and/or enhancers which direct expression of a gene to which they are operatively linked preferentially in muscle cells can be used to create a transgenic animal which expresses a myostatin protein preferentially in muscle tissue. Transgenic animals that include a copy of a myostatin transgene introduced into the germ line of the animal at an embryonic stage can also be used to examine the effect of increased myostatin expression in various tissues.

The pattern and extent of expression of a recombinant molecule of the invention in a transgenic mouse is facilitated by fusing a reporter gene to the recombinant molecule such that both genes are co-transcribed to form a polycistronic mRNA. The reporter gene can be introduced into the recombinant molecule using conventional methods such as those described in Sambrook et al., (Sambrook, 1989). Efficient expression of both cistrons of the polycistronic mRNA encoding the protein of the invention and the reporter protein can be achieved by inclusion of a known internal translational initiation sequence such as that present in poliovirus mRNA. The reporter gene should be under the control of the regulatory sequence of the recombinant molecule of the invention and the pattern and extent of expression of the gene encoding a protein of the invention can accordingly be determined by assaying for the phenotype of the reporter gene. Preferably the reporter gene codes for a phenotype not displayed by the host cell and the phenotype can be assayed quantitatively. Examples of suitable reporter genes include IacZ (β-galactosidase), neo (neomycin phosphotransferase), CAT (chloramphenicol acetyltransferase) dhfr (dihydrofolate reductase), aphIV (hygromycin phosphotransferase), lux (luciferase), uidA (β-glucuronidase). Preferably, the reporter gene is lacZ which codes for β-galactosidase. β-galactosidase can be assayed using the lactose analogue X-gal (5-bromo-4-chloro-3-indolyl-b-D-galactopyranoside) which is broken down by β-galactosidase to a product that is blue in color (Old).

The present invention includes knocking out wild type myostatin in mammals, in order to obtain the desired effect(s) thereof This is particularly true in cattle raised for beef production. It may well prove advantageous to substitute a defective gene (e.g. SEQ ID NO:3 or it genomic analogue) rather than delete the entire sequence of DNA encoding for a protein having myostatin activity. A method of producing a transgenic bovine or transgenic bovine embryo is described in U.S. Pat. No. 5,633,076, issued May 27, 1997, for example.

The transgenic animals of the invention can be used to investigate the molecular basis of myostatin action. For example, it is expected that myostatin mutants in which one or more of the conserved cysteine residues has been deleted would have diminished activity in relation to a wild type myostatin protein in which all such residues are retained. Further, deletion of proteolytic cleavage site would likely result in a mutant lacking biological activity of myostatin.

Transgenesis can be used to inactivate myostatin activity. This could be achieved using either conventional transgenesis, i.e. by injection in fertilized oocytes, or by gene targeting methods using totipotent cell lines such as ES (embryonic stem cells) which can then be injected in oocytes and participate in the development of the resulting organisms or whose nucleus can be transferred into unfertilized oocytes, nucleus transfer or cloning.

It is also possible to create a genetically altered animal in which the double-muscling trait is dominant so that the animal would be more useful in cross-breeding. Further, in a particular aspect, the dominant trait would be male specific. In this way, bulls would be double-muscled but cows would not be. In addition, or alternatively, the trait would also be unexpressed until after birth or inducible. If inducible the trait could be induced after birth to avoid the calving difficulties described above.

There are at least three approaches that can be taken to create a dominant “mh” allele. Because functional myostatin, a member of the TGF-13 superfamily, is a dimer, dominant negative myostatin mutations can be created (Herskowitz et al, 1987; Lopez et al., 1992). According to one method, this is accomplished by mutating the proteolytic processing site of myostatin. To enhance the dominant negative effect, the gene can be put under the control of a stronger promoter such as the CMV promoter or that of a myosin gene, which is tissue specific, i.e., expressed only in skeletal muscle. Alternatively, an antisense sequence of that encoding myostatin could be incorporated into the DNA, so that complementary mRNA molecules are generated, as understood by a person skilled in the art. Optionally, a ribozyme could be added to enhance mRNA breakdown. In another approach, cre recombinase generate/ribozyme approach or the Cre-lox P system could be used (Hoess et al., 1982; Gu et al., 1994).

Male specificity can be achieved by placing the dominant mh alleles on the Y chromosome by homologous recombination.

Inducibility can be achieved by choosing promoters with post-natal expression in skeletal muscle or using inducible systems such at he Tet-On and Tet-Off systems could be used (Gossen et al., 1992; Shockett et al., 1996).

Using conventional transgenesis a gene coding for a myostatin antisense is injected, for example, by inverting the orientation of the myostain gene in front of its natural promoter and enhancer sequences. This is followed by injection of a gene coding for an anti-myostain ribozyme, i.e. an RNA that would specifically bind to endogenous myostain mRNA and destroy it via its “ribozyme” activity.

Also, through gene targeting, a conventional knock-out animal can be generated, specific mutations by gene replacement can be engineered. It is possible to inactivate the myostain gene at a specific developmental time, such as after birth to avoid calving difficulties. As mentioned above, this could be achieved using the Cre-lox P systems in which 1.ox P sides are engineered around the myostain gene by homologous recombination (gene targeting), and mating these animals with transgenic animals having a Cre transgene (coding for the Cre recombinase existing DNA flanked by J oxP sides) under the dependence of a skeletal muscle specific promoter only active after birth. This is done to obtain individuals that would inactivate their myostain gene after birth. As mentioned above, there are also gene targeting systems that allow genes to be turned on and off by feeding an animal with, for example, an antibiotic. In such an instance, one engineers an operator between the promoter of the gene and the gene itself This operator is the target of a repressor which when binding inactivates the gene (for example, the lac operon in E. coli). The repressor is brought into the cell using conventional transgenesis, for example, by injection of the gene coding for the repressor.

Transgenic animals of the invention can also be used to test substances for the ability to prevent, slow or enhance myostatin action. A transgenic animal can be treated with the substance in parallel with an untreated control transgenic animal.

The antisense nucleic acids and oligonucleotides of the invention are useful for inhibiting expression of nucleic acids (e.g. mRNAs) encoding proteins having myostatin activity.

The isolated nucleic acids and antisense nucleic acids of the invention can be used to construct recombinant expression vectors as described previously. These recombinant expression vectors are then useful for making transformant host cells containing the recombinant expression vectors, for expressing protein encoded by the nucleic acids of the invention, and for isolating proteins of the invention as described previously. The isolated nucleic acids and antisense nucleic acids of the invention can also be used to construct transgenic and knockout animals as described previously.

The isolated proteins of the invention are useful for making antibodies reactive against proteins having myostatin activity, as described previously. Alternatively, the antibodies of the invention can be used to isolate a protein of the invention by standard immunoaffinity techniques. Furthermore, the antibodies of the invention, including bispecific antibodies are useful for diagnostic purposes.

Molecules which bind to a protein comprising an amino acid sequence shown in SEQ ID NO:2 can also be used in a method for killing a cell which expresses the protein, wherein the cell takes up the molecule, if for some reason this were desirable. Destruction of such cells can be accomplished by labeling the molecule with a substance having toxic or therapeutic activity. The term “substance having toxic or therapeutic activity” as used herein is intended to include molecules whose action can destroy a cell, such as a radioactive isotope, a toxin (e.g. diphtheria toxin or ricin), or a chemotherapeutic drug, as well as cells whose action can destroy a cell, such as a cytotoxic cell. The molecule binding to the myostatin can be directly coupled to a substance having a toxic or therapeutic activity or may be indirectly linked to the substance. In one example, the toxicity of the molecule taken up by the cell is activated by myostatin protein.

The invention also provides a diagnostic kit for identifying cells comprising a molecule which binds to a protein comprising an amino acid sequence shown in SEQ ID NO:2, for example, for incubation with a sample of tumor cells; means for detecting the molecule bound to the protein, unreacted protein or unbound molecule; means for determining the amount of protein in the sample; and means for comparing the amount of protein in the sample with a standard. Preferably, the molecule is a monoclonal antibody. In some embodiments of the invention, the detectability of the molecule which binds to myostatin is activated by said binding (e.g., change in fluorescence spectrum, loss of radioisotopic label). The diagnostic kit can also contain an instruction manual for use of the kit.

The invention further provides a diagnostic kit for identifying cells comprising a nucleotide probe complementary to the sequence, or an oligonucleotide fragment thereof, shown in SEQ ID NO:1, for example, for hybridization with mRNA from a sample of cells, e.g., muscle cells; means for detecting the nucleotide probe bound to mRNA in the sample with a standard. In a particular aspect, the invention is a probe having a nucleic acid molecule sufficiently complementary with a sequence identified as SEQ ID NO:1, or its complement, so as to bind thereto under stringent conditions. “Stringent hybridization conditions” takes on its common meaning to a person skilled in the art here. Appropriate stringency conditions which promote nucleic acid hybridization, for example, 6× sodium chloride/sodium citrate (SSC) at about 45° C. are known to those skilled in the art. The following examples are found in Current Protocols in Molecular Biology, John Wiley & Sons, NY (1989), 6.3.1-6.3.6: For 50 ml of a first suitable hybridization solution, mix together 24 ml formamide, 12 ml 20× SSC, 0.5 ml 2 M Tris-HCl pH 7.6, 0.5 ml 100× Denhardt's solution, 2.5 ml deionized H₂O, 10 ml 50% dextran sulfate, and 0.5 ml 10% SDS. A second suitable hybridization solution can be 1% crystalline BSA (fraction V), 1 mM EDTA, 0.5 M Na₂HPO₄ pH 7.2, 7% SDS. The salt concentration in the wash step can be selected from a low stringency of about 2× SSC at 50° C to a high stringency of about 0.2× SSC at 50° C. Both of these wash solutions may contain 0.1% SDS. In addition, the temperature in the wash step can be increased from low stringency conditions at room temperature, about 22° C., to high stringency conditions, at about 65° C. The cited reference gives more detail, but appropriate wash stringency depends on degree of homology and length of probe. If homology is 100%, a high temperature (65° C. to 75° C.) may be used. If homology is low, lower wash temperatures must be used. However, if the probe is very short (<100 bp), lower temperatures must be used even with 100% homology. In general, one starts washing at low temperatures (37° C. to 40° C.), and raises the temperature by 3-5° C. intervals until background is low enough not to be a major factor in autoradiography. The diagnostic kit can also contain an instruction manual for use of the kit.

The invention provides a diagnostic kit which can be used to determine the genotype of mammalian genetic material, for example. One kit includes a set of primers used for amplifying the genetic material. A kit can contain a primer including a nucleotide sequence for amplifying a region of the genetic material containing one of the naturally occurring mutations described herein. Such a kit could also include a primer for amplifying the corresponding region of the normal gene that produces functional myostatin. Usually, such a kit would also include another primer upstream or downstream of the region of interest complementary to a coding and/or non-coding portion of the gene. A particular kit includes a primer selected from a non-coding sequence of a myostatin gene. Examples of such primers are provided in Table 3, designated as Exon1-5′, Exon1-3′, Exon2-5′, Exon3-5′ and Exon3-3′. These primers are used to amplify the segment containing the mutation of interest. The actual genotyping is carried out using primers that target specific mutations described herein and that could function as allele-specific oligonucleotides in conventional hybridization, Taqman assays, OLE assays, etc. Alternatively, primers can be designed to permit genotyping by microsequencing.

One kit of primers thus includes first, second and third primers, (a), (b) and (c), respectively. Primer (a) is based on a region containing a myostatin mutation, for example a region of the myostatin gene spanning the nt821del(11) deletion. Primer (b) encodes a region upstream or downstream of the region to be amplified by primer (a) so that genetic material containing the mutation is amplified, by PCR, for example, in the presence of the two primers. Primer (c) is based on the region corresponding to that on which primer (a) is based, but lacking the mutation. Thus, genetic material containing the non-mutated region will be amplified in the presence of primers (b) and (c). Genetic material homozygous for the wild type gene will thus provide amplified products in the presence of primers (b) and (c). Genetic material homozygous for the mutated gene will thus provide amplified products in the presence of primers (a) and (b). Heterozygous genetic material will provide amplified products in both cases.

The invention provides purified proteins having biological activity of myostatin. The terms “isolated” and “purified” each refer to a protein substantially free of cellular material or culture medium when produced by recombinant DNA techniques, or chemical precursors or other chemicals when chemically synthesized. In certain preferred embodiments, the protein having biological activity of myostatin comprises an amino acid sequence identified as SEQ ID NO:2. Furthermore, proteins having biological activity of myostatin that are encoded by nucleic acids which hybridize under stringent conditions, as discussed above, to a nucleic acid comprising a nucleotide sequence identified as SEQ ID NO:1 or SEQ ID NO:7 are encompassed by the invention. Proteins of the invention having myostatin activity can be obtained by expression in a suitable host cell using techniques known in the art. Suitable host cells include prokaryotic or eukaryotic organisms or cell lines, for example, yeast, E. coli, insect cells and COS 1 cells. The recombinant expression vectors of the invention, described above, can be used to express a protein having myostatinI activity in a host cell in order to isolate the protein. The invention provides a method of preparing an purified protein of the invention comprising introducing into a host cell a recombinant nucleic acid encoding the protein, allowing the protein to be expressed in the host cell and isolating and purifying the protein. Preferably, the recombinant nucleic acid is a recombinant expression vector. Proteins can be isolated from a host cell expressing the protein and purified according to standard procedures of the art, including ammonium sulfate precipitation, column chromatography (e.g. ion exchange, gel filtration, affinity chromatography, etc.), electrophoresis, and ultimately, crystallization (see generally, “Enzyme Purification and Related Techniques”, Methods in Enzymology, 22, 233-577 (1971)).

Alternatively, the protein or parts thereof can be prepared by chemical synthesis using techniques well known in the chemistry of proteins such as solid phase synthesis (Merrifield, 1964), or synthesis in homogeneous solution (Houbenwcyl, 1987).

The protein of the invention, or portions thereof, can be used to prepare antibodies specific for the proteins. Antibodies can be prepared which bind to a distinct epitope in an unconserved region of a particular protein. An unconserved region of the protein is one which does not have substantial sequence homology to other proteins, for example other members of the myostatin family or other members of the TGFβ superfamily. Conventional methods can be used to prepare the antibodies. For example, by using a peptide of a myostatin protein, polyclonal antisera or monoclonal antibodies can be made using standard methods. A mammal, (e.g. a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay can be used to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures, thus immortalizing these cells and yielding hybridoma cells. Such techniques are well known in the art. For example, the hybridoma technique originally developed by Kohler and Milstein (Kohler, 1975) as well as other techniques such as the human B-cell hybridoma technique (Kozbor, 1983), the EBV-hybridoma technique to produce human monoclonal antibodies (Cole, 1985), and screening of combinatorial antibody libraries (Huse, 1989). Hybridoma cells can be screened immunochemically for production of antibodies specifically reactive with the peptide, and monoclonal antibodies isolated.

The term antibody as used herein is intended to include fragments thereof which are also specifically reactive with a protein having the biological activity of myostatin, or a peptide fragment thereof Antibodies can be fragmented using conventional techniques and the fragments screened for utility in the same manner as described above for whole antibodies. For example, F(ab′)₂ fragments can be generated by treating antibody with pepsin. The resulting F(ab′)₂ fragment can be treated to reduce disulfide bridges to produce Fab′ fragments.

It is also known in the art to make chimeric antibody molecules with human constant regions. See, for example, Morrison et al., Takeda et al., Cabilly et al., Boss et al., Tanaguchi et al., Teng et al. (Morrison, 1985; Takeda, 1985; Cabilly; Boss; Tanaguchi; Teng, 1982), European Patent Publication 0173494, United Kingdom Patent GB 2177096B, PCT Publication WO92/06193 and EP 0239400. It is expected that such chimeric antibodies would be less immunogenic in a human subject than the corresponding non-chimeric antibody.

Another method of generating specific antibodies, or antibody fragments, reactive against protein having the biological activity of a myostatin protein, or a peptide fragment thereof, is to screen expression libraries encoding immunoglobulin genes, or portions thereof, expressed in bacteria, with peptides produced from the nucleic acid molecules of the present invention. For example, complete Fab fragments, VH regions and FV regions can be expressed in bacteria using phage expression libraries. See for example Ward et al., Huse et al., and McCafferty et al. (Ward, 1989; Huse, 1989; McCafferty, 1990). Screening such libraries with, for example, a myostatin protein can identify immunoglobulin fragments reactive with myostatin. Alternatively, the SCID-hu mouse developed by Genpharm can be used to produce antibodies, or fragments thereof The polyclonal, monoclonal or chimeric monoclonal antibodies can be used to detect the proteins of the invention, portions thereof or closely related isoforms in various biological materials, for example they can be used in an ELISA, radioimmunoassay or histochemical tests. Thus, the antibodies can be used to quantify the amount of a myostatin protein of the invention, portions thereof or closely related isoforms in a sample in order to determine the role of myostatin proteins in particular cellular events or pathological states. Using methods described hereinbefore, polyclonal, monoclonal antibodies, or chimeric monoclonal antibodies can be raised to nonconserved regions of myostatin and used to distinguish a particular myostatin from other proteins.

The polyclonal or monoclonal antibodies can be coupled to a detectable substance or reporter system. The term “coupled” is used to mean that the detectable substance is physically linked to the antibody. Suitable detectable substances include various enzymes, prosthetic groups, fluorescent materials, luminescent materials and radioactive materials. Examples of suitable enzymes include horseradish peroxidase, alkaline phosphatase, β-galactosidase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride and phycoerythrin; an example of a luminescent material includes luminol; and examples of suitable radioactive material include ¹²⁵I; ¹³¹I, ³⁵S and ³H. In a preferred embodiment, the reporter system allows quantitation of the amount of protein (antigen) present.

Such an antibody-linked reporter system could be used in a method for determining whether a fluid or tissue sample of a subject contains a deficient amount or an excessive amount of the protein. Given a normal threshold concentration of such a protein for a given type of subject, test kits could thus be developed.

The present invention allows the skilled artisan to prepare bispecific antibodies and tetrameric antibody complexes. Bispecific antibodies can be prepared by forming hybrid hybridomas (Staerz, 1986a &b).

Compositions of the invention are administered to subjects in a biologically compatible form suitable for pharmaceutical administration in vivo. By “biologically compatible from suitable for administration in vivo” is meant a form of the composition to be administered in which any toxic effects are outweighed by the therapeutic effects of the composition. The term “subject” is intended to include living organisms in which a desired therapeutic response can be elicited, e.g. mammals. Examples of subjects include cattle, human, dogs, cats, mice, rats and transgenic species thereof Administration of a therapeutically active amount of the therapeutic compositions of the present invention is defined as an amount effective, at dosages and for periods of time necessary to achieve the desired result. For example, a therapeutically active amount of a compound that inhibits the biological activity of myostatin protein may vary according to factors such as the age, sex, and weight of the individual, as well as target tissue and mode of delivery. Dosage regimes may be adjusted to provide the optimum therapeutic response. For example, several divided doses may be administered daily or the dose may be proportionally reduced as indicated by the exigencies of the therapeutic situation.

Those skilled in the art will know, or be able to ascertain using no more than routine experimentation, many equivalents to the specific embodiments of the invention described herein. Such equivalents are intended to be encompassed by the following claims.

### REFERENCES

Particulars of references cited above are given below. All of the listed references are incorporated herein by reference.

Barendse, W., S. M. Armitage, L. M. Kossarek, A. Shalom, B. W. Kirkpatrick, A. M. Ryan, D. Clayton, L. Li, H. L. Neibergs, N. Zhang, W. M. Grosse, J. Weiss, P. Creighton, F. McCarthy, M. Ron, A. J. Teale, R. Fries, R. A. McGraw, S. S. Moore, M. Georges, M. Soller, J. E. Womack, and D. J. S. Hetzel. 1994. A genetic linkage map of the bovine genome. Nature Genet. 6: 227-235

Bishop, M. D., S. M. Kappes, J. W. Keele, R. T. Stone, S. L. F. Sunden, G. A. Hawkins, S. Solinas Toldo, R. Fries, M. D. Grosz, J. Yoo, and C. W. Beattie. 1994. A genetic linkage map for cattle. Genetics 136: 619-639.

Boss et al., U.S. Pat. No. 4,816,397.

Cabilly et al. U.S. Pat. No. 4,816,567.

Charlier, C.; Coppieters, W.; Farnir, F.; Grobet, L.; Leroy, P.; Michaux, C.; Mni, M.; Schwers, A.; Vanmanshoven, P.; Hanset, R. & Georges, M. (1995) The mh gene causing double-muscling in cattle maps to bovine chromosome 2. Mammalian Genome 6: 788-792.

Chirgwin, J. M.; Przybyla, A. E.; MacDonald, R. J.; Rutter, W. J. (1979) Isolation of biologically active ribonucleic acid from sources enriched in ribonuclease. Biochemistry 18: 5294-5299.

Cockett, N. E.; Jackson, S. P.; Shay, T. D.; Nielsen, D.; Green, R. D.; Georges, M. (1994). Chromosomal localization of the callipyge gene in sheep (Ovis aries) using bovine DNA markers. Proceedings of the National Academy of Sciences, US, 91: 3019-3023.

Cockett, N. E.; Jackson, S. P.; Shay, T. D.; Farnir, F.; Berghmans, S.; Snowder, G.; Nielsen, D.; Georges, M. (1996). Polar overdominance at the ovine callipyge locus. Science 273: 236-238.

Cole et al. (1985). Monoclonal Antibodies in Cancer Therapy. Allen R. Bliss, Inc.

Collins, F. S. 1995. Positional cloning moves from perditional to traditional. Nature Genet. 9: 347-350.

Cornelis, F.; Hashimoto, L.; Loveridge, J.; MacCarthy, A.; Buckle, V.; Julier, C.; Bell, J. (1992). Identification of a CA repeat at the TCRA locus using YACs: a general method for generating highly polymorphic markers at chosen loci. Genomics 13: 820-825.

Cottingham, R. W.; Idury, R. M.; Schäffer, A. A. (1993). Faster sequential genetic linkage computations. Am. J. Hum. Genet. 53: 252-263.

Culley, G. (1807). Observations on livestock. 4th ed., (London, G. Woodfall). Fisher, S. R.; Beever, J. E.; Lewin, H. A. (1996). Genetic mapping of COL3AI to bovine chromosome 2. Mammalian Genome 8: 76-77.

Fuji, J.; Otsu, K.; Zorzato, F.; Deleon, S.; Khanna, V. K.; Weiler, J. E. O'Brien, P. J.; MacLennan, D. H. (1991). Identification of a mutation in the porcine ryanodine receptor associated with malignant hyperthermia. Science 253: 448-451.

Georges, M.; Andersson, L. (1996). Livestock genomics comes of age. Genome Research 6: 907-921.

Georges, M.; Nielsen, D.; Mackinnon, M.; Mishra, A.; Okimoto, R.; Pasquino, A. T.; Sargeant, L. S.; Sorensen, A.; Steele, M. R.; Zhao, X.; Womack, J. E.; Hoeschele, I. (1995). Mapping quantitative trait loci controlling milk production by exploiting progeny testing. Genetics 139: 907-920.

Gossen, M. & Bujard, H. (1992). Tight control of gene expression in mammalian cells by tetracycline-responsive promotors. Proceedings of the National Academy of Sciences, USA,89: 5547-5551.

Grobet, L.; Royo Martin, L. J.; Poncelet, D.; Pirottin, D.; Brouwers, B.; Riquet, J.; Schoeberlein, A.; Dunner, S.; Ménissier, F.; Massabanda, J.; Fries, R.; Hanset, R.; Georges, M. (1997) A deletion in the myostatin gene causes double-muscling in cattle. Nature Genetics 17: 71-74.

Gu, H.; Marth, J. D.; Orban, P. C.; Mossmann, H.; Rajewsky, K. (1994). Deletion of a DNA polymerase beta gene segment in T cells using cell type-specific gene targetting. Science 265: 103-106.

Hanset, R. and Michaux, C. (1985a). On the genetic determinism of muscular hypertrophy in the Belgian White and Blue cattle breed. I. Experimental data. G6nét. Sél. Evol. 17: 359-368.

Hanset, R. and Michaux, C. (1985b). On the genetic determinism of muscular hypertrophy in the Belgian White and Blue cattle breed. II. Population data. Génét. Sél. Evol. 17: 369-386.

Hanset, R. (1991). The major gene of muscular hypetrophy in the belgian Blue Cattle Breed. In Breeding for Disease Resistance in Farm Animals, Owen, Axford, eds. C.A.B. International, pp.467-478.

Herskowitz, I. (1987). Functional inactivation of genes by dominant negative mutations. Nature 329:219-222. Hogan, B. et al., (1986). A Laboratory Manual, Cold Spring Harbor, N.Y., Cold Spring Harbor Laboratory.

Hoess, R. H.; Ziese, M.; Sternberg, N. (1982). P1 site-specific recombination: nucleotide sequence of the recombining sites. Proc.Natl.Acad.Sci.USA 79: 3398-3402.

Houbenwcyl, (1987). Methods of Organic Chemistry, ed. E. Wansch. Vol. 15 I and II. Thieme, Stuttgart.

Hudson et al. (1995) Science 270:1945-1954 with supplementary data from the Whitehead Institute/MIT Center for Genome Research, Human Genetic Mapping Project, data release 11.9 (May 1997)

Hunter, K.; Riba, L.; Schalkwyk, L.; Clark, M.; Resenchuk, S.; Beeghly, A.; Su, J.; Tinkov, F.; Lee, P.; Ramu, E.; Lehrach, H. and Housman, D. (1996). Toward the Construction of Integrated Physical and Genetic Maps of the Mouse Genome Using Interspersed Repetitive Sequence PCR (IRS\NPCR) Genomics. Genome Research 6: 290-299.

Huse et al., (1989). Science 246: 1275-1281.

Kambadur, R.; Sharma, M.; Smith, T. P. L.; Bass, J. J. (1997). Mutations in myostatin (GDF8) in double-muscled Belgian Blue Cattle. Genome Research 7: 910-916.

Kappes, S. M.; Keele, J. W.; Stone, R. T.; McGraw, R. A.; Sonstegard, T. S.; Smith, T. P. L.; Lopez-Corrales, N. L. and Beattie, C. W. (1997). A Second-Generation Linkage Map of the Bovine Genome. Genome Research 7: 235-249.

Kennett, R. (1979). Cell fusion. Methods Enzymol. 58: 345-359.

Kohler and Milstein. (1975). Nature 256: 495-497.

Kozbor et al. (1983). Immunol. Today 4: 72.

Lathrop, M.; Lalouel, J. M. (1984). Easy calculations of lodscores and genetic risk on small computers. American Journal of Human Genetics 36: 460-465.

Lenstra, J. A.; van Boxtel, J. A. F.; Zwaagstra, K. A.; Schwerin, M. (1993). Short interspersed nuclear element (SINE) sequences of the Bovidae. Animal Genetics 24: 33-39.

Libert, F.; Lefort, A.; Okimoto, R.; Georges, M. (1993) Construction of a bovine genomic library of large yeast artificial chromosome clones. Genomics 18: 270-276.

Lopez, A. R.; Cook, J.; Deininger, P. L.; Derynck, R. (1992). Dominant negative mutants of trnasforming growth factor-betal inhibit the secretation of different transforming growth factor beta isoforms. Molecular and Cellular biology 12(4): 1674-1679.

Lyons, A. L.; Laughlin, T. F.; Copeland, N. G.; Jenkins, N. A.; Womack, J. E.; O'Brien, S. J. (1996). Comparative Anchor tagged Sequences for Integrative mapping of Mammalian Genomes. Nature Genetics 15: 47-56.

McPherron, A. C.; Lee, S. -J. (1996). The transforming growth factor β superfamily. In Growth Factors and Cytokines in Health and Disease, Volume 1B, pages 357-393. JAI press Inc.

McPherron, A. C.; Lawler, A. M.; Lee, S. -J. (1997). Regulation of skeletal muscle mass in mice by a new TGFβ superfamily member. Nature 387: 83-90.

Ménissier, F. (1982). Present state of knowledge about the genetic determination of muscular hypertrophy or the double muscled trait in cattle. in Current Topics in Veterinary Medicine and Animal Science, vol. 16: Muscle hypertrophy of genetic origin and its use to improve beef production, pp. 387-428. Ed. King and Ménissier, Martinus Nijhoff.

Merrifield, (1964]. J. Am. Chem. Assoc. 85: 2149-2154.

McCafferty et al.) (1990). Nature 348: 552-554.

Mirski, S. and Cole, S. P. C. (1989). Antigens associated with multidrug resistance in H69AR, a small cell lung cancer cell line. Cancer Res. 49: 5719-5724.

Morrison etal., (1985). Proceedings of the National Academy of Sciences, USA, 81: 6851.

O'Brien, S. J.; Womack, J. E.; Lyons, L. A.; Moore, K. J.; Jenkins, N. A.; Copeland, N. G. (1993). Anchored reference loci for comparative genome mapping in mammals. Nature Genetics 3: 103-112.

Old, R. W. and Primrose, S. B., In: Principles of Gene Manipulation. An Introduction to Genetic Engineering, 4th ed. Oxford University Press. 63-66.

Pell, J. M.; Flint, D. J.; (1997). In: Milk Composition, Production and Biotechnology, Ed. Welch et al., Chapter 19.

Sambrook, J., Fritsch E. F. and Maniatis, T. (1989). Molecular Cloning: A Laboratory Manual. Cold Spring Harbor Lab Press, Cold Spring Harbor, New York.

Shockett, P. E.; Schatz, D. G. (1996). Diverse strategies for tetracycline-regulated inducible gene expression. Proceedings of the National Academy of Sciences, USA, 93: 5173-5176.

Solinas-Toldo, S.; Lengauer, C; Fries, R. (1995). Comparative genome map of man and cattle. Genomics 27: 489-496.

Staerz & Bevan (1986a). Proceedings of the National Academy of Sciences, USA, 83: 1453.

Staerz & Bevan (1986b). Immunology Today 7: 241.

Stewart, A. J., Canitrot, Y., Baracchini, E., Dean, N. M., Deeley, R. G., and Cole, S. P. C. (1996). Reduction of Expression of the multidrug resistance protein (MRP) in human tumor cells by antisense phophorothioate oligonucleotides. Biochem. Pharamcol. 51: 461-469.

Takeda et al., (1985). Nature 314: 452.

Tanaguchi et al., European Patent Publication EP171496.

Teng, et al.. (1982) Meth. Enzymol. 92: 3-16.

Varga, L.; Szabo, G.; Darvasi, A.; Müller, G.; Sass, M.; Soller, M. (1997). Inheritance and mapping of compact (Cmpt), a new mutation causing hypermuscularity in mice. Genetics, in the press.

Walter, M. A.; Spillett, D. J.; Thomas, P.; Weissenbach, J.; Goodfellow, P. N. (1994). A method for constructing radiation hybrid maps of whole genomes. Nature Genetics 7:22-28. Ward et al, (1989). Nature 341: 544-546.

What is claimed is:

1. A method of increasing muscle mass of a mammal having muscle cells in which myostatin is expressed, the method comprising administering to the mammal an effective amount of a nucleic acid molecule substantially complementary to at least a portion of mRNA encoding the myostatin and being of sufficient length to sufficiently reduce expression of the myostatin to increase the muscle mass.

2. The method of claim 1 wherein the mammal is bovine.

3. A method of increasing muscle mass of a mammal, the method comprising administering to the mammal an effective amount of a nucleic acid molecule having ribozyme activity and a nucleotide sequence substantially complementary to at least a portion of mRNA encoding myostatin and being of sufficient length to bind selectively thereto to sufficiently reduce expression of the myostatin so as to increase the muscle mass.

4. The method of claim 3 wherein the mammal is bovine.

5. A diagnostic kit, for determining the presence of muscular hyperplasia in a mammal from which a sample containing DNA of the mammal has been obtained, the kit comprising:
first and second primers for amplifying the DNA, the primers being complementary to nucleotide sequences of the DNA upstream and down stream, respectively, of a mutation in the portion of the DNA encoding myostatin which results in muscular hyperplasia of the mammal, wherein at least one of the nucleotide sequences is selected to be from a non-coding region of the myostatin gene.

6. The diagnostic kit of claim 5, further comprising a third primer complementary to a naturally occurring mutation of a coding portion of the myostatin gene.

7. A diagnostic kit, for determining the genotype of a sample of mammalian genetic material, the kit comprising:
a pair of primers for amplifying a portion of the genetic material corresponding to a nucleotide sequence which encodes at least a portion of a myostatin protein, wherein a first of the primers includes a nucleotide sequence sufficiently complementary to a mutation of SEQ ID NO:1 to prime amplification of a nucleic acid molecule containing the mutation, the mutation being selected from the group of mutations resulting from: (a) deletion of 11 nucleotides beginning at nucleotide 821 of the coding portion of SEQ ID NO:1; (b) deletion of 7 nucleotides beginning at nucleotide 419 of the coding sequence and insertion of the sequence AAGCATACAA in place thereof; (c) deletion of nucleotide 204 of the coding sequence and insertion of T in place thereof; (d) deletion of nucleotide 226 of the coding sequence and insertion of T in place thereof; and (e) deletion of nucleotide 313 of the coding sequence and insertion of A in place thereof; and combinations thereof.

8. The diagnostic kit of claim 7 wherein a second of the pair of primers is located entirely upstream or entirely downstream of the selected mutation or mutations.

9. The diagnostic kit of claim 8 wherein a first said primer spans mutation (a) and further comprising a third primer which is sufficiently complementary to the nucleotide sequence identified as SEQ ID NO:11 to prime amplification of a nucleic acid molecule containing SEQ ID NO:1.

10. The diagnostic kit of claim 8 wherein a first said primer is sufficiently complementary to the inserted sequence of mutation (b) to prime amplification of a nucleic acid molecule containing mutation (b) and further comprising a third primer which is sufficiently complementary to the sequence corresponding to the 7 nucleotide deletion of mutation (b) to prime amplification of a nucleic acid molecule containing the 7 nucleotide deletion of mutation (b).

11. The diagnostic kit of claim 8 wherein a first said primer spans mutation (c) and further comprising a third primer which is sufficiently complementary to the sequence spanning the corresponding region lacking mutation (c) to prime amplification of a nucleic acid molecule lacking mutation (c).

12. The diagnostic kit of claim 8 wherein a first said primer spans mutation (d) and further comprising a third primer which is sufficiently complementary to the sequence spanning the corresponding region lacking mutation (d) to prime amplification of a nucleic acid molecule lacking mutation (d).

13. The diagnostic kit of claim 8 wherein a first said primer spans mutation (e) and further comprising a third primer which is sufficiently complementary to the sequence spanning the corresponding region lacking mutation (e) to prime amplification of a nucleic acid molecule lacking mutation (e).

14. A method for determining the presence of muscular hyperplasia in a bovine animal, the method comprising:
obtaining a sample of material containing DNA from a said animal; and
ascertaining whether DNA having a nucleotide sequence encoding a protein having biological activity of myostatin is present,
wherein the absence of DNA having said nucleotide sequence indicates the presence of muscular hyperplasia in the animal.

15. The method of claim 14 wherein ascertaining whether DNA having a nucleotide sequence encoding a protein having biological activity of myostatin includes amplifying the DNA in the presence of primers based on a nucleotide sequence encoding a protein having biological activity of myostatin.

16. The method of claim 15 wherein DNA of a said bovine animal not displaying muscular hyperplasia has a nucleotide sequence which is capable of hybridizing with a nucleic acid molecule having the sequence identified as SEQ ID NO:1 under stringent hybridization conditions.

17. The method of claim 14, wherein ascertaining whether DNA having a nucleotide sequence encoding a protein having biological activity of myostatin is present includes amplifying the DNA in the presence of primers based on a nucleotide sequence encoding the N-terminal and the C-terminal, respectively, of the protein having biological activity of myostatin.

18. The method of claim 14, wherein ascertaining whether DNA having a nucleotide sequence encoding a protein having biological activity of myostatin is present includes amplifying the DNA in the presence of first and second primers based on first and second nucleotide sequences encoding spaced apart regions of the protein, wherein said regions flank a mutation known to naturally occur and which when present in both alleles of a said animal results in said muscular hyperplasia.

19. The method of claim 18 wherein a DNA of said animal not displaying muscular hyperplasia contains a nucleotide sequence which hybridizes under stringent conditions with a nucleotide sequence encoding a protein having a sequence identified as SEQ ID NO:2 and the coding sequence of DNA of a said animal displaying muscular hyperplasia is known to contain an 11-base pair deletion beginning at base pair no. 821, and said first primer is selected to be upstream of the codon encoding glutamic acid no. 275 and the second primer is selected to be downstream of the codon encoding aspartic acid no. 274.

20. The method of claim 14 wherein a DNA of said animal not displaying muscular hyperplasia contains a nucleotide sequence which hybridizes under stringent conditions with a nucleotide sequence encoding a protein having a sequence identified as SEQ ID NO:2 and the coding sequence of DNA of a said animal displaying muscular hyperplasia is known to contain an 11-base pair deletion beginning at base pair no. 821, and said primer is selected to span the nucleotide sequence including base pair nos. 820 and 821 of the DNA sequence containing said deletion.

21. The method of claim 19 wherein the animal is of a breed selected from Belgian Blue, Asturiana, Parthenaise and Rubia Gallega.

22. The method of claim 20 wherein the animal is a breed selected from Belgian Blue, Asturiana, Parthenaise and Rubia Gallega.

23. The method of claim 14 wherein ascertaining whether DNA having a nucleotide sequence encoding a protein having biological activity of myostatin is present includes amplifying the DNA in the presence of a primer containing at least a portion of a mutation known to naturally occur and which when present in both alleles of a said animal results in said muscular hyperplasia.

24. A method for determining the presence of muscular hyperplasia in a bovine animal, the method comprising:
obtaining a sample of material containing DNA from a said animal; and
ascertaining whether DNA having a mutation as defined in claim 7 is present; and
ascertaining whether DNA having a nucleotide sequence encoding a protein having biological activity of myostatin is present,
wherein the absence of DNA having said nucleotide sequence and presence of a said mutation indicates the presence of muscular hyperplasia in the animal.

25. A method for determining the presence of muscular hyperplasia in a bovine animal, the method comprising:
obtaining a sample of the animal containing mRNA; and
ascertaining whether an mRNA encoding a protein having biological activity of myostatin is present in the sample,
wherein the absence of said mRNA indicates the presence of muscular hyperplasia in the animal.

26. The method of claim 25 wherein the sample is of muscle tissue or wherein the tissue is skeletal muscle tissue.

27. The method of claim 25 wherein ascertaining whether mRNA having a nucleotide sequence encoding a protein having biological activity of myostatin includes amplifying the mRNA in the presence of primers substantially complementary to the nucleotide sequence encoding the protein.

28. The method of claim 27 wherein mRNA of a said bovine animal not displaying muscular hyperplasia has a nucleotide sequence which is capable of hybridizing with a nucleic acid molecule having the sequence identified as SEQ ID NO:1 under stringent hybridization conditions.

29. The method of claim 25, wherein ascertaining whether mRNA having a nucleotide sequence encoding a protein having biological activity of myostatin is present includes amplifying the mRNA in the presence of primers substantially complementary to a nucleotide sequence encoding the N-terminal and the C-terminal, respectively, of the protein having biological activity of myostatin.

30. The method of claim 25, wherein ascertaining whether mRNA having a nucleotide sequence encoding a protein having biological activity of myostatin is present includes amplifying the mRNA in the presence of first and second primers substantially complementary to first and second nucleotide sequences encoding spaced apart regions of the protein, wherein said regions flank a mutation known to naturally occur and which when present in both alleles of a said animal results in said muscular hyperplasia.

31. The method of claim 30 wherein an mRNA of said animal not displaying muscular hyperplasia contains a nucleotide sequence which hybridizes under stringent conditions with a nucleotide sequence encoding a protein having a sequence identified as SEQ ID NO:2 and the coding sequence of DNA of a said animal displaying muscular hyperplasia is known to contain an 11-base pair deletion beginning at base pair no. 821, and said first primer is selected to be upstream of the codon encoding glutamic acid no. 275 and the second primer is selected to be downstream of the codon encoding aspartic acid no. 274.

32. The method of claim 25 wherein ascertaining whether mRNA having a nucleotide sequence encoding a protein having biological activity of myostatin is present includes amplifying the mRNA in the presence of a primer containing a nucleotide sequence complementary to at least a portion of a mutation known to naturally occur in a said animal and which when present in both alleles of a said animal results in said muscular hyperplasia.

33. The method of claim 32 wherein an mRNA of said animal not displaying muscular hyperplasia contains a nucleotide sequence which hybridizes under stringent conditions with a nucleotide sequence encoding a protein having a sequence identified as SEQ ID NO:2 and the coding sequence of DNA of a said animal displaying muscular hyperplasia is known to contain an 11-base pair deletion beginning at base pair no. 82 1, and said primer is selected to span the deleted portion.

34. The method of claim 31 wherein the animal is of a breed selected from Belgian Blue, Asturiana, Parthenaise and Rubia Gallega.

35. A method for determining the presence of muscular hyperplasia in a mammal, the method comprising:
obtaining a sample of material containing DNA from the mammal; and
ascertaining whether a sequence of the DNA encoding (a) a protein having biological activity of myostatin, is present, and whether a sequence of the DNA encoding (b) an allelic protein lacking the activity of (a), is present;
wherein the absence of (a) and the presence of (b) indicates the presence of muscular hyperplasia in the mammal.

36. The method of claim 35 wherein (b) contains a naturally occurring mutation responsible for the lack of activity.

37. The method of claim 35 wherein the mammal is a human.

38. The method of claim 37 wherein ascertaining whether a sequence of the DNA encoding (a) is present, and whether a sequence of the DNA encoding (b) is present includes amplifying the DNA in the presence of primers based on a nucleotide sequence encoding a protein having biological activity of myostatin.

39. The method of claim 38 wherein said primers are based on the sequence identified as SEQ ID NO:7.

40. A method for determining the presence of muscular hyperplasia in a mammal, the method comprising:
obtaining a sample of material containing mRNA from the mammal; and
ascertaining whether a sequence of the mRNA encoding (a) a protein having biological activity of myostatin, is present, and whether a sequence of the mRNA encoding (b) a protein at least partially encoded by a truncated nucleotide sequence corresponding to substantially the sequence of the mRNA and lacking the activity of (a), is present;
wherein the absence of (a) and the presence of (b) indicates the presence of muscular hyperplasia in the mammal.

41. The method of claim 40 wherein the mRNA encoding (a) and the truncated sequence correspond to alleles of DNA of the mammal.

42. The method of claim 40 wherein the mammal is human.

43. The method of claim 42 wherein ascertaining whether a sequence of the mRNA encoding (a) is present, and whether a sequence of the mRNA encoding (b) is present includes amplifying the mRNA in the presence of a pair of primers complementary to a nucleotide sequence encoding a protein having biological activity of myostatin.

44. The method of claim 43 wherein each said primer contains a truncated nucleotide sequence substantially complementary to a portion of the sequence identified as SEQ ID NO:7.

45. The method of claim 44 wherein the truncated sequence contains at least 50 consecutive nucleotides substantially corresponding to about 10, or between about 10 and 20, or between about 20 and 30, or between about 30 and 40, or between about 40 and 50 consecutive nucleotides of SEQ ID NO:7.

46. A method for determining the presence of muscular hyperplasia in a mammal, the method comprising:
obtaining a tissue sample of containing mRNA of the mammal; and
ascertaining whether an mRNA encoding a mutant type myostatin protein lacking biological activity of myostatin is present,
wherein the presence of a said mRNA encoding a mutant type myostatin protein indicates the presence of muscular hyperplasia in the mammal.

47. The method of claim 46 wherein the mutant type myostatin protein lacing biological activity is encoded by a naturally occurring allele of DNA encoding the mRNA.

48. A method for determining the presence of double muscling in a bovine animal, the method comprising:
obtaining a sample of material containing DNA from the animal; and
ascertaining whether the DNA contains the nucleotide coding sequence identified as SEQ ID NO:11,
wherein absence of the sequence indicates double muscling in the animal.

49. The method of claim 34 wherein the animal is of a breed selected from Belgian Blue, Asturiana, Parthenaise and Rubia Gallega.

50. A method for determining the myostatin genotype of a mammal, comprising:
obtaining a sample of material containing nucleic acid of the mammal, wherein the nucleic acid is uncontaminated by heterologous nucleic acid;
ascertaining whether the sample contains a (i) nucleic acid molecule encoding a protein having biological activity of myostatin; and
ascertaining whether the sample contains an (ii) allelic nucleic acid molecule encoding a protein lacking biological activity of myostatin.

51. The method of claim 50 wherein the mammal is human and (i) comprises a nucleic acid sequence substantially homologous with the sequence identified as SEQ ID NO:7.

52. A purified protein having biological activity of myostatin, and having an amino acid sequence identified as SEQ ID NO:2, or a conservatively substituted variant thereof.

53. An isolated nucleic acid molecule encoding a protein of claim 52.

54. An isolated nucleic acid molecule comprising a DNA molecule having the nucleotide sequence identified as SEQ ID NO:1 or which varies from the sequence due to the degeneracy of the genetic code, or a nucleic acid strand capable of hybridizing with at least one said nucleic acid molecule under stringent hybridization conditions.

55. Isolated mRNA transcribed from DNA having a sequence which corresponds to a nucleic acid molecule according to claim 54.

56. Isolated DNA having a sequence according to claim 54 in a recombinant cloning vector.

57. A microbial cell containing and expressing heterologous DNA which is complementary a nucleic acid molecule of claim 54.

58. A transfected cell line which expresses a protein of claim 52.

59. A process for producing the protein of claim 52 comprising:
preparing a DNA fragment including a nucleotide sequence which encodes said protein;
incorporating the DNA fragment into an expression vector to obtain a recombinant DNA molecule which includes the DNA fragment and is capable of undergoing replication;
transforming a host cell with said recombinant DNA molecule to produce a transformant which can express said protein;
culturing the transformant to produce said protein; and
recovering said protein from resulting cultured mixture.

60. A method of increasing muscle mass in a mammal, comprising administering an effective amount of an antibody to myostatin to said mammal.

61. A method of increasing muscle mass in a mammal, comprising raising an autoantibody to the myostatin the in the mammal.

62. The method of claim 61 wherein raising the autoantibody includes administering a protein having myostatin activity to the mammal.

63. A method of increasing muscle mass in a mammal in need thereof, comprising administering to the mammal an effective amount of an antisense nucleic acid or oligonucleotide substantially complementary to at least a portion of the sequence identified as SEQ ID NO:1 or SEQ ID NO:5, or SEQ ID NO:7.

64. The method of claim 63 wherein the portion is at least 5 nucleotide bases in length.

65. The method of claim 64 wherein the mammal is a bovine and the sequence is the sequence identified as SEQ ID NO:1.

66. A method of increasing muscle mass in a mammal, comprising administering to the mammal an effective amount of an antibody to the myostatin.

67. A probe comprising a nucleic acid molecule sufficiently complementary with a sequence identified as SEQ ID NO:1, or its complement, so as to bind thereto under stringent conditions.

68. The probe of claim 67 wherein the sequence is between about 8 and about 1195 nucleotides in length, or between about 15 and 1195 nucleotides in length, or between about 25 and 1195 nucleotides in length, or between about 35 and 1195 nucleotides in length, or between about 45 and 1195 nucleotides in length, or between about 55 and 1195 nucleotides in length, or between about 65 and 1195 nucleotides in length, or between about 75 and 1195 nucleotides in length, or between about 75 and 1195 nucleotides in length, or between about 85 and 1195 nucleotides in length, or between about 95 and 1195 nucleotides in length, or between about 105 and 1195 nucleotides in length, or between about 115 and 1195 nucleotides in length.

69. A method for identifying a nucleotide sequence of a mutant gene encoding a myostatin protein of a mammal displaying muscular hyperplasia, the method comprising:
obtaining a sample of material containing DNA from the mammal; and
probing the sample using a nucleic acid probe based on a nucleotide sequence of a known gene encoding myostatin in order to identify nucleotide sequence of the mutant gene.

70. The method of claim 69, wherein the probe is based on a nucleotide sequence of a non-coding region of the gene.

71. The method of claim 70 wherein the probe is based on SEQ ID NO:54.

72. The method of claim 71 wherein the probe is at least 8 nucleic acids in length.

73. The method of claim 69, wherein the step of probing the sample includes exposing the DNA to the probe under hybridizing conditions and further comprising isolating hybridized nucleic acid molecules.

74. The method of claim 73, further comprising the step of sequencing isolated DNA.

75. The method of claim 69, wherein the mammal is a bovine mammal and the probe is based on a said nucleotide sequence identified as SEQ ID NO:1.

76. The method of claim 74, further comprising the step of isolating and sequencing a cDNA or mRNA encoding the complete mutant myostatin protein.

77. The method of claim 71, further comprising the step of isolating and sequencing a functional wild type myostatin from a said mammal not displaying muscular hyperplasia.

78. The method of claim 76, further comprising comparing the complete coding sequence of the complete mutant myostatin protein with, if the coding sequence for a functional wild type myostatin from a said mammal is previously known, (1) the known sequence, or if the coding sequence for a functional wild type myostatin from a said mammal is previously unknown, (2) the sequence determined according to claim 74 or claim 77, to determine the location of any mutation in the mutant gene.

79. A method for determining the myostatin genotype of a mammal, wherein wild type myostatin of the mammal is substantially that of claim 78, comprising:
obtaining a sample of material containing DNA from the mammal; and
ascertaining whether the DNA contains a said mutation determined according to claim 78.

80. A method for determining the myostatin genotype of a mammal, wherein wild type myostatin of the mammal is substantially that of claim 78, comprising:
obtaining a sample of material containing mRNA from the mammal; and
ascertaining whether the mRNA contains a said mutation determined according to claim 78.

81. A primer composition useful for the detection of a nucleotide sequence encoding a myostatin comprising a first nucleic acid molecule based on a nucleotide sequence located upstream of a said mutation determined according to claim 78 and a second nucleic acid molecule based on a nucleotide sequence located downstream of the mutation.

82. A probe comprising a nucleic acid molecule based on a nucleotide sequence of claim 74 or claim 76 and spanning a said mutation determined according to claim 78.

83. A transgenic mammal having a phenotype characterized by muscular hyperplasia, said phenotype being conferred by a transgene contained in the somatic and germ cells of the mammal, the transgene encoding a myostatin protein having a dominant negative mutation.

84. The transgenic mammal of claim 83 wherein the mammal is male and non-human and the transgene is located on the Y chromosome.

85. The transgenic mammal of claim 83 wherein the mammal is bovine and the transgene is located to be under the control of a promoter which normally a promoter of a myosin gene.

86. A transgenic mammal having a phenotype characterized by muscular hyperplasia, said phenotype being conferred by a transgene having a sequence antisense to that encoding a myostatin protein of the mammal.

87. The transgenic mammal of claim 86 wherein the mammal is bovine and the transgene is located on the Y chromosome.

88. The transgenic mammal of claim 86 wherein the transgene further comprises a sequence which when transcribed obtains an mRNA having ribozyme activity.

89. A transgenic non-human mammal having a phenotype characterized by muscular hyperplasia, said phenotype being inducible and being conferred by a myostatin gene flanked by J oxP sides and a Cre transgene under the dependence of an inducible promoter.

90. A transgenic non-human male mammal having a phenotype characterized by muscular hyperplasia, said phenotype being conferred by a myostatin gene flanked by J oxP sides and a Cre transgene located on the Y chromosome.

91. A method for determining whether a sample of mammalian genetic material is capable of a conferring a phenotype characterized by muscular hyperplasia, comprising ascertaining whether the genetic material contains a nucleotide sequence encoding a protein having biological activity of myostatin, wherein the absence of said sequence indicates the presence of muscular hyperplasia in the animal.

92. A transgenic bovine having a genome lacking a gene encoding a protein having biological activity of myostatin.

93. A transgenic mouse having a genome containing a gene encoding a human protein having biological activity of myostatin or containing a gene encoding a bovine protein having biological activity of myostatin.

94. A transgenic bovine having a gene encoding a bovine protein having biological activity of myostatin and heterologous nucleotide sequence antisense to the gene.

95. A transgenic bovine of claim 94, further comprising a gene encoding a nucleic acid sequence having ribozyme activity and in transcriptional association with the nucleotide sequence antisense to the gene.


SEQUENCE LISTING (54 sequences)

SEQ ID NO: 1 - DNA, 1196 bp, Bos taurus
aggaaaaagtaagaacaagggaaaagattgtattgattttaaaaccatgcaaaaactgcaaatctctgtttatatttacctatttatgctgattgttgctggcccagtggatctgaatgagaacagcgagcagaaggaaaatgtgcaaaaagaggggctgtgtaatgcatgtttgtggagggaaaacactacatcctcaagactagaagccataaaaatccaaatcctcagtaaacttcgcctggaaacagctcctaacatcagcaaagatgctatcagacaacttttgcccaaggctcctccactcctggaactgattgatcagttcgatgtccagagagatgccagcagtgacggctccttggaagacgatgactaccacgccaggacggaaacggtcattaccatgcccacggagtctgatcttctaacgcaagtggaaggaaaacccaaatgttgcttctttaaatttagctctaagatacaatacaataaactagtaaaggcccaactgtggatatatctgaggcctgtcaagactcctgcgacagtgtttgtgcaaatcctgagactcatcaaacccatgaaagacggtacaaggtatactggaatccgatctctgaaacttgacatgaacccaggcactggtatttggcagagcattgatgtgaagacagtgttgcagaactggctcaaacaacctgaatccaacttaggcattgaaatcaaagctttagatgagaatggccatgatcttgctgtaaccttcccagaaccaggagaagatggactgactccttttttagaagtcaaggtaacagacacaccaaaaagatctaggagagattttgggcttgattgtgatgaacactccacagaatctcgatgctgtcgttaccctctaactgtggattttgaagcttttggatgggattggattattgcacctaaaagatataaggccaattactgctctggagaatgtgaatttgtatttttgcaaaagtatcctcatacccatcttgtgcaccaagcaaaccccagaggttcagccggcccctgctgtactcctacaaagatgtctccaattaatatgctatattttaatggcgaaggacaaataatatacgggaagattccagccatggtagtagatcgctgtgggtgttcatgagtctatatttgggttcataagc

SEQ ID NO: 2 - protein, 375 aa, Bos taurus
MQKLQISVYIYLFMLIVAGPVDLNENSEQKENVEKEGLCNACLWRENTTSSRLEAIKIQILSKLRLETAPNISKDAIRQLLPKAPPLLELIDQFDVQRDASSDGSLEDDDYHARTETVITMPTESDLLTQVEGKPKCCFFKFSSKIQYNKLVKAQLWIYLRPVKTPATVFVQILRLIKPMKDGTRYTGIRSLKLDMNPGTGIWQSIDVKTVLQNWLKQPESNLGIEIKALDENGHDLAVTFPEPGEDGLTPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGEGQIIYGKIPAMVVDRCGCS

SEQ ID NO: 3 - DNA, 1240 bp, Bos taurus
aggaaaaagtaagaacaagggaaaagattgtattgattttaaaaccatgcaaaaactgcaaatctctgtttatatttacctatttatgctcattgttgctggcccagtggatctgaatgagaacagcgagcagaaggaaaatgtggaaaaagaggggctgtgtaatgcatgtttgtggagggaaaacactacatcctcaagactagaagccataaaaatccaaatcctcagtaaacttcgcctggaaacagctcctaacatcagcaaagatgctatcagacaacttttgcccaaggctcctccactcctggaactgattgatcagttcgatgtccagagagatgccagcagtgacggctccttggaagacgatgactaccacgccaggacggaaacggtcattaccatgcccacggagtctgatcttctaacgcaagtggaaggaaaacccaaatgttgcttctttaaatttagctctaagatacaatacaataaactagtaaaggcccaactgtggatatatctgaggcctgtcaagactcctgcgacagtgtttgtgcaaatcctcagactcatcaaacccatgaaagacggtacaaggtatactggaatccgatctctgaaacttgacatgaacccaggcactggtatttggcagagcattgatgtgaagacagtgttgcagaactggctcaaacaacctgaatccaacttaggcattgaaatcaaagctttagatgagaatggccatgatcttgctgtaaccttcccagaaccaggagaagatggactgactccttttttagaagtcaaggtaacagacacaccaaaaagatctaggagagattttgggcttgattgtgacagaatctcgatgctgtcgttaccctctaactgtggattttgaagcttttggatgggattggattattgcacctaaaagatataaggccaattactgctctggagaatgtgaatttgtatttttgcaaaagtatcctcatacccatcttgtgcaccaagcaaaccccagaggttcagccggcccctgctgtactcctacaaagatgtctccaattaatatgctatattttaatggcgaaggacaaataatatacgggaagattccagccatggtagtaaatcgctgtgggtgttcatgaggtctatatttggttcatagcttcctcaaacatggaaggtcttcccctcaacaattttgaaactgttgaaattatgt

SEQ ID NO: 4 - protein, 286 aa, Bos taurus
MQKLQISVYIYLFMLIVAGPVDLNENSEQKENVEKEGLCNACLWRENTTSSRLEAIKIQILSKLRLETAPNISKDAIRQLLPKAPPLLELIDQFDVQRDASSDGSLEDDDYHARTETVITMPTESDLLTQVEGKPKCCFFKFSSKIQYNKLVKAQLWIYLRPVKTPATVFVQILRLIKPMKDGTRYTGIRSLKLDMNPGTGIWQSIDVKTVLQNWLKQPESNLGIEIKALDENGHDLAVTFPEPGEDGLTPFLEVKVTDTPKRSRRDFGLDCDRISMLSLPSNCGF

SEQ ID NO: 5 - DNA, 2676 bp, Mus musculus
gtctctcggacggtacatgcactaatatttcacttggcattactcaaaagcaaaaagaagaaataagaacaagcgaaaaaaaaagattgtgctgatttttaaaatgatgcaaaaactgcaaatgtatgtttatatttacctcttcatgctgattgctgctggcccagtggatctaaatgagggcagtgagagagaagaaaatgtggaaaaagaggggctgtgtaatgcatgtgcgtggagacaaaacacgaggtactccagaatagaagccataaaaattcaaatcctcagtaagctgcgcctggaaacagctcctaacatcagcaaagatgctataagacaacttctgccaagagcgcctccactccgggaactgatcgatcagtacgacgtccagagggatgacagcagtgatggctctttggaagatgacgattatcacgctaccacggaaacaatcattaccatgcctacagagtctgactttctaatgcaagcggatggcaagcccaaatgttgcttttttaaatttagctctaaaatacagtacaacaaagtagtaaaagcccaactgtggatatatctcagacccgtcaagactcctacaacagtgtttgtgcaaatcctgagactcatcaaacccatgaaagacggtacaaggtatactggaatccgatctctgaaacttgacatgagcccaggcactggtatttggcagagtattgatgtgaagacagtgttgcaaaattggctcaaacagcctgaatccaacttaggcattgaaatcaaagctttggatgagaatggccatgatcttgctgtaaccttcccaggaccaggagaagatgggctgaatccctttttagaagtcaaggtgacagacacacccaagaggtcccggagagactttgggcttgactgcgatgagcactccacggaatcccggtgctgccgctaccccctcacggtcgattttgaagcctttggatgggactggattatcgcacccaaaagatataaggccaattactgctcaggagagtgtgaatttgtgtttttacaaaaatatccgcatactcatcttgtgcaccaagcaaaccccagaggctcagcaggcccttgctgcactccgacaaaaatgtctcccattaatatgctatattttaatggcaaagaacaaataatatatggcaaaattccagccatggtagtagaccgctgtgggtgctcatgagctttgcattaggttagaaacttcccaagtcatggaaggtcttcccctcaatttcgaaactgtgaattcaagcaccacaggctgtaggccttgagtatgctctactaacgtaagcacaagctacagtgtatgaactaaaagagagaatagatgcaatggttggcattcaaccaccaaaataaaccatactataggatgttgtatgatttccagagtttttgaaatagatggagatcaaattacatttatgtccatatatgtatattacaactacaatctaggcaaggaagtgagagcacatcttgtggtctgctgagttaggagggtatgattaaaaggtaaagtcttatttcctaacagtttcacttaatatttacagaacaatctatatgtagcctttgtaaagtgtaggattgttatcatttaaaaacatcatgtacacttatatttgtattgtatacttggtaagataaaattccacaaagtaggaatggggcctcacatacacattgccattcctattataattggacaatccaccacggtgctaatgcagtgctcaatggctcctactggacctctcgatagaacactctacaaagtacgagtctctctctcccttccaggtgcatctccacacacacagcactaagtgttcaatgcattttctttaaggaaagaagaatctttttttctagaggtcaactttcagtcaactctagcacagcgggagtgactgctgcatcttaaaaggcagccaaacagtattcattttttaatctaaatttcaaaatcactgtctgcctttatcacatggcaattttgtggtaaaataatggaaatgactggttctatcaatattgtataaaagactctgaaacaattacatttatataatatgtatacaatattgttttgtaaataagtgtctccttttatatttactttggtatatttttacactaatgaaatttcaaatcattaaagtacaaagacatgtcatgtatcacaaaaaaggtgactgcttctatttcagagtgaattagcagattcaatagtggtcttaaaactctgtatgttaagattagaaggttatattacaatcaatttatgtattttttacattatcaacttatggtttcatggtggctgtatctatgaatgtggctcccagtcaaatttcaatgccccaccattttaaaaattacaagcattactaaacataccaacatgtatctaaagaaatacaaatatggtatctcaataacagctacttttttattttataatttgacaatgaatacatttcttttatttacttcagttttataaattggaactttgtttatcaaatgtattgtactcatagctaaatgaaattatttcttacataaaaatgtgtagaaactataaattaaagtgttttcacatttttgaaaggc

SEQ ID NO: 6 - protein, 376 aa, Mus musculus
MMQKLQMYVYIYLFMLIAAGPVDLNEGSEREENVEKEGLCNACAWRQNTRYSRIEAIKIQILSKLRLETAPNISKDAIRQLLPRAPPLRELIDQYDVQRDDSSDGSLEDDDYHATTETIITMPTESDFLMQADGKPKCCFFKFSSKIQYNKVVKAQLWIYLRPVKTPTTVFVQILRLIKPMKDGTRYTGIRSLKLDMSPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 7 - DNA, 2215 bp, Homo sapiens
  CDS (1): Unsure
ngattttctaatgcaagtggatggaaaacccaaatgttgcttctttaaatttagctctaaaatacaatacaataaagtagtaaaggcccaactatccatatatttgagacccgtcgagactcctacaacagtgtttgtgcaaatcctgagactcatcaaacctatgaaagacggtacaaggtatctggaatccgatctctgaaacttgacatgaacccaggcactggtatttgggcaganattgatgtgaagacactgttgcaaaattggctcaaacaacctgaatccaacttaggcattgaaataaaagctttacatgagaatggtcatgatcttgctgtaaccttcccaggaccaggaagaagatgggctgaatcccttttttaagaaggtcaaggtaacagacacaccaaaaagattccagaagggattttgggtcttgactggtgatgagcactcaacagaatcacgatcctgtcgttaccccctaactggtggattttgaagcctttgggatgggattggatatcgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagcgatggtagtagaccgctgtgggtgctcagcgatggtagtagaccgctgtgggtgctcttttcaagctgtgaaattaagtaccacaggctataggcctagagtatgctacagtcacttaagcataagctacagtatgtaaactaaaagggggaangggaatatatgcaatggttggcatttaaccatccaaacaaatcataccagaaagttttatgatttccanagttttttnaggcnagaaaggaggagtcaaantttcantcttatggtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatttcggcacaggtnaaacacttgaatttatattgtatggtagtatacttggtaagataaaattccacaaaaatagggatggtgcagcatatgcaatttccattcctattataattgacacagtacattaacaatccatgccaacggtgctaatacgataggctgannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaaatctcaacgttccattattttaatacttgcaaaaacattactaagtataccaaaataattgactctattatctgaaatgaagaataaactgatgctatctcaacaataactgttacttttattttataatttgataatgaatatatttctgcatttatttacttctgttttgtaaattgggattttgttaatcaaatttattgtactatgactaaatgaaattatttcttacatctaatttgtagaaacagtataagttatattaaagtgttttcacatttttttgaaagac

SEQ ID NO: 8 - protein, 375 aa, Homo sapiens
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 9 - DNA, 22 bp, Artificial sequence
  Artificially Synthesized Primer Sequence
ggcccaactatggatatatttg

SEQ ID NO: 10 - DNA, 21 bp, Artificial sequence
  Artificially Synthesized Primer Sequence
ggtcctgggaaggttacagca

SEQ ID NO: 11 - DNA, 11 bp, bos taurus
atgaacactcc

SEQ ID NO: 12 - DNA, 26 bp, Artificial sequence
  Artificially Synthesized Primer Sequence
cagcaaagtccttaatggtaacaagc

SEQ ID NO: 13 - DNA, 23 bp, Artificial sequence
  Artificially Synthesized Primer Sequence
gggtcactgaagaaaacgtcctg

SEQ ID NO: 14 - DNA, 23 bp, Artificial sequence
  Artificially Synthesized Primer Sequence
ccccatattatggagatgaaccg

SEQ ID NO: 15 - DNA, 23 bp, Artificial sequence
  Artificially Synthesized Primer Sequence
agttcaggatggcagaatttcag

SEQ ID NO: 16 - DNA, 23 bp, Artificial sequence
  Artificially Synthesized Primer Sequence
gcaaactgggyggragcaagacc

SEQ ID NO: 17 - DNA, 24 bp, Artificial sequence
  Artificially Synthesized Primer Sequence
ttsttcctgggcttttattgagac

SEQ ID NO: 18 - DNA, 25 bp, Artificial sequence
  Artificially Synthesized Primer Sequence
aagccwgatttctgcttyttggaag

SEQ ID NO: 19 - DNA, 25 bp, Artificial sequence
  Artificially Synthesized Primer Sequence
tgccmaggcahccrccrtacttgaa

SEQ ID NO: 20 - DNA, 19 bp, Artificial sequence
  Artificially Synthesized Primer Sequence
ggtcgtcctacaccagaag

SEQ ID NO: 21 - DNA, 22 bp, Artificial sequence
  Artificially Synthesized Primer Sequence
ggttgacattgtcaagaacaag

SEQ ID NO: 22 - DNA, 24 bp, Artificial sequence
  Artificially Synthesized Primer Sequence
tctcmaaagtcgtctgtgacaatc

SEQ ID NO: 23 - DNA, 23 bp, Artificial sequence
  Artificially Synthesized Primer Sequence
tgytcrttttctttcagagttgc

SEQ ID NO: 24 - DNA, 23 bp, Artificial sequence
  Artificially Synthesized Primer Sequence
rctggtcctcttcacctcagaac

SEQ ID NO: 25 - DNA, 23 bp, Artificial sequence
  Artificially Synthesized Primer Sequence
acattgtcvgttccaaagccaag

SEQ ID NO: 26 - DNA, 21 bp, Artificial sequence
  Artificially Synthesized Primer Sequence
aggtycgggtgacdgtgctkc

SEQ ID NO: 27 - DNA, 23 bp, Artificial sequence
  Artificially Synthesized Primer Sequence
tggrtacatgagytccaccttgc

SEQ ID NO: 28 - DNA, 23 bp, Artificial sequence
  Artificially Synthesized Primer Sequence
agctgcargtatwcctacaayct

SEQ ID NO: 29 - DNA, 23 bp, Artificial sequence
  Artificially Synthesized Primer Sequence
gtyccrttgctcytctcrttgyc

SEQ ID NO: 30 - DNA, 25 bp, Artificial sequence
  Artificially Synthesized Primer Sequence
aactgtatattgagagcctaccatg

SEQ ID NO: 31 - DNA, 24 bp, Artificial sequence
  Artificially Synthesized Primer Sequence
cacaccttagcgactaaaccacca

SEQ ID NO: 32 - DNA, 25 bp, Artificial sequence
  Artificially Synthesized Primer Sequence
ctacctaacagaatgattttgtaag

SEQ ID NO: 33 - DNA, 25 bp, Artificial sequence
  Artificially Synthesized Primer Sequence
agtgttcttgcctagagaatcccag

SEQ ID NO: 34 - DNA, 25 bp, Artificial sequence
  Artificially Synthesized Primer Sequence
acattctctcaccaatatgacatac

SEQ ID NO: 35 - DNA, 25 bp, Artificial sequence
  Artificially Synthesized Primer Sequence
taagtcaccattacatccttagaac

SEQ ID NO: 36 - DNA, 25 bp, Artificial sequence
  Artificially Synthesized Primer Sequence
gctgtaagaatcttcattaagcact

SEQ ID NO: 37 - DNA, 25 bp, Artificial sequence
  Artificially Synthesized Primer Sequence
cctgatacatgctaaggttaaaaac

SEQ ID NO: 38 - DNA, 25 bp, Artificial sequence
  Artificially Synthesized Primer Sequence
aggcatacatctggagagaaacatg

SEQ ID NO: 39 - DNA, 25 bp, Artificial sequence
  Artificially Synthesized Primer Sequence
cagaggagcctagcaggctaccgtc

SEQ ID NO: 40 - DNA, 25 bp, Artificial sequence
  Artificially Synthesized Primer Sequence
cagcaggtctgttgaagtgtatcag

SEQ ID NO: 41 - DNA, 25 bp, Artificial sequence
  Artificially Synthesized Primer Sequence
agtggtagcattcacaggtagccag

SEQ ID NO: 42 - DNA, 20 bp, Artificial sequence
  Artificially Synthesized Primer Sequence
cagtccatggcaccataaag

SEQ ID NO: 43 - DNA, 22 bp, Artificial sequence
  Artificially Synthesized Primer Sequence
tccgttagtactggctaattgc

SEQ ID NO: 44 - DNA, 20 bp, Artificial sequence
  Artificially Synthesized Primer Sequence
ctgaattggctccaaaggcc

SEQ ID NO: 45 - DNA, 20 bp, Artificial sequence
  Artificially Synthesized Primer Sequence
aaacagaagtccagggctgc

SEQ ID NO: 46 - DNA, 21 bp, Artificial sequence
  Artificially Synthesized Primer Sequence
tcagtctccaggagagaaaac

SEQ ID NO: 47 - DNA, 20 bp, Artificial sequence
  Artificially Synthesized Primer Sequence
ctctgccctggggatgattg

SEQ ID NO: 48 - DNA, 28 bp, Artificial sequence
  Artificially Synthesized Primer Sequence
aatgtatgtttatatttacctgttcatg

SEQ ID NO: 49 - DNA, 25 bp, Artificial sequence
  Artificially Synthesized Primer Sequence
acagtgtttgtgcaaatcctgagac

SEQ ID NO: 50 - DNA, 25 bp, Artificial sequence
  Artificially Synthesized Primer Sequence
caatgcctaagttggattcaggttg

SEQ ID NO: 51 - DNA, 25 bp, Artificial sequence
  Artificially Synthesized Primer Sequence
cttgctgtaaccttcccaggaccag

SEQ ID NO: 52 - DNA, 23 bp, Artificial sequence
  Artificially Synthesized Primer Sequence
tcccatccaaaggcttcaaaatc

SEQ ID NO: 53 - DNA, 25 bp, Artificial sequence
  Artificially Synthesized Primer Sequence
atactcwaggcctayagcctgtggt

SEQ ID NO: 54 - DNA, 5790 bp, bos taurus
gcggccgcccgggcaggtatcgaaagtttcacatataaagatgaataagatctaagtgtatatgttattgttaataaagtttttaatttttcgaatgtcacatacagcctttattattcatagatttattccttttaagaagtagtcaaatgaatcagctcacccttgactgtaacaaaatactgtttggtgacttgtgacagacagggttttaacctctgacagcgagattcattgtggagcaagagccaatcacagatcccgacgacacttgtctcatcaaagttggaatataaaaagccacttggaatacagtataaaagattcactggtgtggcaagttgtctctagactgggcaggcattaacgtttggcttggcgttactcaaaagcaaaagaaaagtaaaaggaagaagtaagaacaagggaaaagattgtattgattttaaaaccatgcaaaaactgcaaatctctgtttatatttacctatttatgctgattgttgctggcccagtggatctgaatgagaacagcgagcagaaggaaaatgtggaaaaagaggggctgtgtaatgcatgtttgtggagggaaaacactacatcctcaagactagaagccataaaaatccaaatcctcagtaaacttcgcctggaaacagctcctaacatcagcaaagatgctatcagacaacttttgcccaaggctcctccactcctggaactgattgatcagttcgatgtccagagagatgccagcagtgacggctccttggaagacgatgactaccacgccaggacggaaacggtcattaccatgcccacggagtgtgagtagtcctgctggtgcaaagcaacgactctgctgactgctgttctagtgttcatgaaaaaccgatctattttcaggctcttttaacaagctgctggcttgtatgtaaggaggaggggaaagagcttttttcaagatttcatgagaaatagaccaatgagactgaaagctgctactttatttgtttccttagagagctaaaaagctaaaaatcaaaaatgaaatgcttgcatagcattcatgttatatagtttagtatgacaactataacatgtttatgttttcacagcttaatgctaccaaggtaaaggattgggaaacagtatcagcaatgtgaaaaatttacatcaaatttcctaattgcatttggttgcctgaaatatgcatttataataacaggttttttttttttcattaataaaagagaaaggaagaaatctgtagaggttgaagcctatctgggcatttgctgaacacttagaatgacttctgttattcaaaactatttctcatagggtttttatggtcttcacagagtatctaattttgaaagctattagagtggaaaggataaaagaatattcttaataaacttaatgtattagtaagagcaataaggaagtaaacacagcatagtgaaaaatcatgagctaatcagcagaaaattctaagaaataaacattttaattacaaagttccacttataccctgaccatggtactattgttgagagtaccttgtctgcacatatctaggaggcacatgcttaataaccttctaaaatattattgtattcctcataggagggagaactattacctatatgtagtacctatgttgtttctgaaagataatatgtttcatgtatttctgttgcagtcacttcaaacctatactcaaggaaagggagacaggcatctcaacagagaaggcatgaccagaaagagttttgtgccatgtgtctgcgatcttgctttatacagggctctacccactttaaactggactcaaaacagtttcaaaatactgctttttcttattaagtaactagtttataaggcaacaaataaatttcctttaagactgtgctatcagataatcctggaatagatttgccttacttataaacaatcttgagaaaacaaaaaggcaagaaattgctaagtgcttctgcttacaatgacagcctggccctaaagacaatgttttctaagttttgaaacagcttgaatacaacatctaagttttggtgctaattacctgctagtttttttatttttttcctttaaaaggctgtcccagcgtcctaacataacagatgcactatattttctgctaattcccgaggctcagttagttgctcactgtgtcttgtccccaggtaattcaggcctgggggaagggttccttcctccagactgattggtacagctgctcagtaagtgtaactactcagattcccaaagaattctaagtggatgttcttccacagtgtctcttgttctctctaatcatcatcattttaaaatttcatccacttttcattccttaatagaattttccttagtccacagttctctggaaaggaagtaggcttctcataacagctgaaaaaacatatacctaaaagattctgaaaagctgtaataactgttatacttgatattttgctgttatgaatgaaatgctacatatttttccattttaaaagactaaatatgcacacattattccaattaaaaaatgttcatagattgatatggaggtgttcgttcatttttcataaaaatgatcttagtaactttttttcttattcatttatagctgatcttctaacgcaagtggaaggaaaacccaaatgttgcttctttaaatttagctctaagatacaatacaataaactagtaaaggcccaactgtggatatatctgaggcctgtcaagactcctgcgacagtgtttgtgcaaatcctgagactcatcaaacccatgaaagacggtacaaggtatactggaatccgatctctgaaacttgacatgaacccaggcactggtatttggcagagcattgatgtgaagacagtgttgcagaactggctcaaacaacctgaatccaacttaggcattgaaatcaaagctttagatgagaatggccatgatcttgctgtaaccttcccagaaccaggagaagatggactggtaagtgattactgaaaataacatgctaaaaaccttgttatgtgtttattcataatgtgaatgaatagtagtgaaaaataactaccagtttcctgtgcttataagccagacaaaggcaccttaccccagtggtagccctgtactcaataaaagtaggtgtcccatttcacatcctatgaaacactctcttgatactttgactttgcatgaggatttaaaagaaaaaaagttataccatggtccttaagtttttagggaattctttggaattgagaatgaaatataaaatgctttccgttgatgtgctacatgattatataaataaaaacatgaagtcttcacagtggattctagtactcacccaacaacacattttttcccccagaagagtgaccaatttgttaaaattcttttgcttaataaggcagaaaaatgaactctacaagttataattaaaataaaatgcttttacttatagaaattaactagatatatgttcaggtttatatactattaaatatactatatttaagatctctcatgataaatatgttccttgttttatagactattgatgcactgatgtatatgtggattactttgtgaattacccctggtaaaattaaaaatttcaggctagttaacttgtactacttagctattttctgaactgtcttactgttctttaacaggagttaacttaggtaatgtcaactaatttaatataaagtcaaacagaaaataatgccttatatattataaaaattaataaaaaaccattttaaaatctagtataagtttagagctactcactcttctggcttatctatgcttgtatttacttctgttttcaaaaaattttttaatgtgaccataccttttatttccagttattgatataatttacaacaaaagattatacttgcaagctttatagtttttaaatggtcttatttgtagtgaatatcatatctaaatgatatctaaatgtaaagtaaatcatacctaaatgaaaacatattctttaagtcattataaaattttccaggtgatcaatttttctttaaatatactacataaaatgttattgactcccaaaatgatgttattttgtataatcttaaataccaataattaccaggtctattttggttttagtgtaggataaaaaagaatgtgttcttttttctaggtagcattttaatgatcaaagttggtgacgtgacagaggtcttaagtattattaaacagatgattaataagatgtattcctcagacttttccatataaaaggaaaaatgtctcaaattcatgaaaagattggtacaggaggaggattagcaaattgtagtttaaatatctgaatggaaacactttttagtgaaagaataaagggaatatcattgtatcttcttctgagtctgtgcctctctctcttggagttagtctttccaaccctatatacttaccactatcttcatccctctaccttcctttttcccattacatctgtgcagtactgggtggcaactattgtgtttcggtgttaatatccaagtttccctgaataagaccaagtgaatggaggatgaatgagtatacctatccctccaggggtcatcagacatatttagccaccatatttaatcaataagcaggaagacataagctagccttgtccttcttctttcctccctgctcctttctcttctcttccccctctccctttactgtcatccatcagtattttcagagcatctattatgtgtcaggcattcagatactcaaacggaggaaaacaagaataaacaagacaaagatctgaccacaggggaatccctatggctactgtagacttttgagccataaaggaagaatcaagcctagtgtaaatgaaaattccttaatgctgtgccttttaaaaagaaatgtgacataagcaaaatgattagtttctttctttaataatgagtccttgaggtaggagagtgttttgggatctattattaactcttctttcctttccatacagactccttttttagaagtcaaggtaacagacacaccaaaaagatctaggagagattttgggcttgattgtgatgaacactccacagaatctcgatgctgtcgttaccctctaactgtggattttgaagcttttggatgggattggattattgcacctaaaagatataaggccaattactgctctggagaatgtgaatttgtatttttgcaaaagtatcctcatacccatcttgtgcaccaagcaaaccccagaggttcagccggcccctgctgtactcctacaaagatgtctccaattaatatgctatattttaatggcgaaggacaaataatatacgggaagattccagccatggtagtagatcgctgtgggtgttcatgaggtctatatttggttcatagcttcctcaaacatggaaggtcttcccctcaacaattttgaaactgtgaaattatgtaccacaggctataagcctagagtatgctacagtcacttaagcacaagctacagtatatgagctaaaaagagagaatatatgcaatggttggcatttaaccatccaaacaaatcgtataataaaaagttttatgatttccagagtttttgaactaggagatcaaattccatttatgttgaaatatattacaacacatgcaggtgaatgaaagcaattctccttgtcttctggtgaattaaaggagtatgctttaaaatctatttctctacagtttc